(19) United States
(12) Patent Application Publication
(10) Pub. No.: US 2007/0208015 A1
(43) Pub. Date: Sep. 6, 2007

(54) 5-Morpholinylmethylthiophenyl Pharmaceutical Compounds As P38 MAP Kinase Modulators

(75) Inventors: Adrian Liam Gill, Cambridge (GB); Maria Grazia Carr, Luton (GB); John Francis Lyons, London (GB); Neil Thomas Thompson, Cambridge (GB); David Charles Rees, Cambridge (GB)

Correspondence Address: HESLIN ROTHENBERG FARLEY & MESITI PC, 5 COLUMBIA CIRCLE, ALBANY, NY, 12203, US

(73) Assignee: ASTEX THERAPEUTICS, LTD., Cambridge (UK)

(21) Appl. No.: 10/599,931

(22) PCT Filed: Apr. 13, 2005
(86) PCT No.: PCT/GB05/01350

§ 371 Date: Apr. 19, 2007

Related U.S. Application Data
(60) Provisional application No. 60/561,600, filed on Apr. 13, 2004.
(60) Provisional application No. 60/618,830, filed on Oct. 14, 2004.

(30) Foreign Application Priority Data
Apr. 13, 2004 (GB) 0408239.2

(51) Int. Cl. A61K 31/5377; C07D 413/14

(52) U.S. Cl. 514/231.5; 544/140; 544/146; 514/235.5

(57) ABSTRACT

The invention provides compounds of the formula (I) or a salt, solvate or N-oxide thereof, wherein: R¹ and R² are the same or different and each is selected from hydrogen, saturated C₁₋₃ hydrocarbyl, halogen and cyano; X is selected from C═O, C═S, C(═O)NH, C(═S)NH, C(═O)O, C(═O)S, C(═S)O and C(═S)S; R³ is selected from aryl and heteroaryl groups each having from 5 to 12 ring members and being unsubstituted or substituted by one or more substituent groups R¹⁰ as defined in the claims; R⁴ and R⁵ are the same or different and are selected from hydrogen and methyl; or one of R⁴ and R⁵ is selected from hydroxymethyl and ethyl and the other is hydrogen; and R⁶ and R⁷ are the same or different and are selected from hydrogen and methyl. The compounds of the formula (I) hayed activity as p38 MAP kinase and Taf kinase inhibitors.

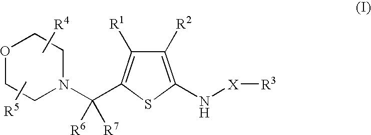

This invention relates to thiophene amide compounds, pharmaceutical compositions containing the compounds, the therapeutic uses of the compounds and novel chemical intermediates. The invention also relates to the use of the compounds as inhibitors or modulators of p38 MAP kinase activity, their use as inhibitors of raf kinases and their use as agents for preventing angiogenesis.

Compounds of the invention are also presented for use in the treatment or prophylaxis of disease states or conditions mediated by p38 MAP kinase or raf kinases and for use in the treatment or prophylaxis of cancers.

### BACKGROUND OF THE INVENTION

Protein kinases constitute a large family of structurally related enzymes that are responsible for the control of a wide variety of signal transduction processes within the cell (Hardie, G. and Hanks, S. (1995) The Protein Kinase Facts Book. I and II, Academic Press, San Diego, Calif.). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks, S. K., Hunter, T., FASEB J, 9:576-596 (1995); Knighton, et al., Science, 253:407-414 (1991); Hiles, et al., Cell, 70:419-429 (1992); Kunz, et al., Cell, 73:585-596 (1993); Garcia-Bustos, et al., EMBO J., 13:2352-2361 (1994)).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signalling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth and differentiation factors, etc.), cell cycle events, environmental or nutritional stresses, etc. The appropriate protein kinase functions in signalling pathways to activate or inactivate (either directly or indirectly), for example, a metabolic enzyme, regulatory protein, receptor, cytoskeletal protein, ion channel or pump, or transcription factor. Disruption of intracellular signal transduction due to defective control of protein phosphorylation has been implicated in a number of diseases, including, for example, inflammation, cancer, allergy/asthma, disease and conditions of the immune system, disease and conditions of the central nervous system, and angiogenesis.

P38 MAP Kinases

The mitogen-activated protein (MAP) kinase family consists of a series of structurally related proline-directed serine/threonine kinases that are activated either by growth factors (such as EGF) and phorbol esters (ERK), or by IL-1, TNF or stress (p38, JNK). These kinases mediate the effects of numerous extracellular stimuli on a wide array of biological processes, such as cell proliferation, differentiation and death. Three groups of mammalian MAP kinases have been studied in detail: the extracellular signal-regulated kinases (ERK), the c-Jun NH₂-terminal kinases (JNK) and the p38 MAP kinases.

There are five known human isoforms of p38 MAP kinase, p38α, p38β, p38β2, p38γ and p38δ. The p38 kinases, which are also known as cytokine suppressive anti-inflammatory drug binding proteins (CSBP), stress activated protein kinases (SAPK) and RK, are responsible for phosphorylating (Stein et al., Ann. Rep. Med Chem., 31, 289-298 (1996)) and activating transcription factors (such as ATF-2, MAX, CHOP and C/ERPb) as well as other kinases (such as MAPKAP-K/3 or MK2/3), and are themselves activated by physical and chemical stress (e.g. UV, osmotic stress), pro-inflammatory cytokines and bacterial lipopolysaccharide (LPS) (Herlaar, E & Brown, Z., Molecular Medicine Today, 5: 439-447 (1999)). The products of p38 phosphorylation have been shown to mediate the production of inflammatory cytokines, including TNF and IL-1, and cyclooxygenase-2 (COX-2). Each of these cytokines has been implicated in numerous disease states and conditions. IL-1 and TNF are also known to stimulate the production of other proinflammatory cytokines such as IL-6 and IL-8.

Interleukin-1 (IL-1) and Tumor Necrosis Factor (TNF) are biological substances produced by a variety of cells, such as monocytes or macrophages. IL-1 has been demonstrated to mediate a variety of biological activities thought to be important in immunoregulation and other physiological conditions such as inflammation (e.g. Dinarello, et al., Rev. Infect. Disease, 6: 51 (1984)). The myriad of known biological activities of IL-1 include the activation of T helper cells, induction of fever, stimulation of prostaglandin or collagenase production, neutrophil chemotaxis, induction of acute phase proteins and the suppression of plasma iron levels.

There are many disease states in which excessive or unregulated IL-1 production is implicated in exacerbating and/or causing the disease. These include rheumatoid arthritis (Arend et al., Arthritis & Rheumatism 38(2): 151-160, osteoarthritis, endotoxemia and/or toxic shock syndrome, other acute or chronic inflammatory disease states such as the inflammatory reaction induced by endotoxin or inflammatory bowel disease; tuberculosis, atherosclerosis, Hodgkin's disease (Benharroch et al., Euro. Cytokine Network 7(1): 51-57), muscle degeneration, cachexia, psoriatic arthritis, Reiter's syndrome, gout, traumatic arthritis, rubella arthritis, acute synovitis and Alzheimer's disease. Evidence also links IL-1 activity to diabetes and pancreatic B cells (Dinarello, J. Clinical Immunology, 5: 287-297 (1985)). Because inhibition of p38 leads to inhibition of IL-1 production, it is envisaged that p38 inhibitors will be useful in the treatment of the above listed diseases.

Excessive or unregulated TNF production has been implicated in mediating or exacerbating a number of diseases including rheumatoid arthritis (Maini et al., APMIS, 105(4): 257-263), rheumatoid spondylitis, osteoarthritis, gouty arthritis and other arthritic conditions; sepsis, septic shock, endotoxic shock, gram negative sepsis, toxic shock syndrome, adult respiratory distress syndrome, cerebral malaria, chronic pulmonary inflammatory disease, silicosis, pulmonary sarcoisosis, bone resorption diseases, reperfusion injury, graft vs. host reaction, allograft rejections, fever and myalgias due to infection, such as influenza, herpes simplex virus type-1 (HSV-1), HSV-2, cytomegalovirus (CMV), varicella-zoster virus (VZV), Epstein-Barr virus (EBV), human herpes virus-6 (HHV-6), HHV-7, HHV-8, pseudorabies, rhinotracheitis and cachexia secondary to infection or malignancy, cachexia secondary to acquired immune deficiency syndrome (AIDS), AIDS, ARC (AIDS related complex), keloid formation, scar tissue formation, Crohn's disease, ulcerative colitis, or pyresis. Because inhibition of p38 leads to inhibition of TNF production, it is envisaged that p38 inhibitors will be useful in the treatment of the above listed diseases.

Interleukin-8 (IL-8) is a chemotacetic factor produced by several cell types including mononuclear cells, fibroblasts, endothelial cells, and keratinocytes. Its production from endothelial cells is induced by IL-1, TNF, or lipopolysaccharide (LPS). IL-8 stimulates a number of functions in vitro. It has been shown to have chemoattractant properties for neutrophils, T-lymphocytes, and basophils. In addition it induces histamine release from basophils from both normal and atopic individuals as well as lysozomal enzyme release and respiratory burst from neutrophils. IL-8 has also been shown to increase the surface expression of Mac-1 (CD 11 blCD 18) on neutrophils without de novo protein synthesis; this may contribute to increased adhesion of the neutrophils to vascular endothelial cells. Many diseases are characterized by massive neutrophil infiltration. Conditions associated with an increased in IL-8 production (which is responsible for chemotaxis of neutrophils into the inflammatory site) would benefit from treatment with compounds which are suppressive of IL-8 production. Recently Chronic Obstructive Pulmonary Disease (COPD) has been linked to raised levels of IL-8 and neutrophil infiltration of the lung (Barnes et al., Curr. Opin. Pharmacol., 1: 242-7 (2001)). Other conditions linked to IL-8 include acute respiratory distress syndrome (ARDS), asthma, pulmonary fibrosis and bacterial pneumonia. IL-1 and TNF affect a wide variety of cells and tissues and these cytokines as well as other leukocyte derived cytokines are important and critical inflammatory mediators of a wide variety of disease states and conditions. The inhibition of these cytokines is of benefit in controlling, reducing and alleviating many of these disease states.

Inhibition of signal transduction via p38, which in addition to IL-1, TNF and IL-8 described above is also required for the synthesis and/or action of several additional pro-inflammatory proteins (i.e., IL-6, GM-CSF, COX-2, collagenase and stromelysin), is expected to be a highly effective mechanism for regulating the excessive and destructive activation of the immune system. This expectation is supported by the potent and diverse anti-inflammatory activities described for p38 kinase inhibitors (Badger, et al., J. Pharm. Exp. Thera., 279: 1453-1461(1996); Griswold, et al., Pharmacol. Comm., 7: 323-229 (1996)).

raf Kinase

Many compounds of the invention have been found to be active as inhibitors of raf kinase.

Raf kinase is a key downstream target for the ras GTPase and mediates the activation of the MAP kinase cascade consisting of raf-MEK-ERK. Activated ERK is a kinase that subsequently targets a number of proteins responsible for mediating amongst other things the growth, survival and transcriptional functions of the pathway. These include the transcription factors ELK1, C-JUN, the Ets family including Ets1, Ets2 and Ets7, and the FOS family. The ras-raf-MEK-ERK signal transduction pathway is activated in response to many cell stimuli including growth factors such as EGF, PDGF, KGF etc. Because the pathway is a major target for growth factor action the activity of raf-MEK-ERK has been found to be upregulated in many factor dependent tumours. The observation that about 20% of all tumours have undergone an activating mutation in one of the ras proteins indicates that the pathway is more broadly important in tumorigenesis. There is growing evidence that activating mutations in other components of the pathway also occur in human tumours. This is true for the raf kinases.

There are 3 closely related isoforms of raf, (A-raf, B-raf, and c-raf-1) which are activated by ras and translocate to the membrane as a consequence of this interaction. Recent evidence indicates that mutational activation of B-raf is found in a number of different tumours including >65% of malignant melanomas, >10% of colorectal cancers (Rajagopalan, H. et al., Nature, 418, 934(2002)), ovarian cancers (Singer, G., et al., J. Natl. Cancer Inst., 95, 484-486 (2003)) and papillary thyroid cancers (Brose, M., et al., Cancer Res., 62, 6997-7000(2002); Cohen, Y., et al., Invest. Ophthalmol. Vis. Sci., 44, 2876-2878(2003)). A range of different B-raf mutations have been identified in different tumours with the most common being a V599E mutation in the so-called activation loop of the kinase domain (Davies, H., et al., Nature, 417, 949-954 (2002).

Other mutations of B-raf found associated with human cancers may not necessarily activate B-raf kinase directly but do up-regulate the activity of the ras-raf-MEK-ERK pathway by mechanisms which are not fully understood but may involve cross talk with other raf isoforms, such as A-raf (Wan, P., et al., Cell, 116, 855-867 (2004)). In such cases inhibition of raf activity would remain a beneficial aim in cancer treatment.

In addition to link between B-raf and certain tumour types, there is a significant amount of evidence to indicate a more broad inhibition of raf kinase activity could be beneficial as an antitumour therapy. Blocking the pathway at the level of B-raf would be effective at counteracting the upregulation of this pathway caused by tumourigenic ras mutations and also in tumours responding to growth factor action via this pathway. Genetic evidence in Drosophila and C. elegans indicates that raf homologues are essential for ras dependent actions on differentiation (Dickson, B., et al., Nature, 360, 600-603 (1993)). Introduction of constitutively active MEK into NIH3T3 cells can have a transforming action whilst expression of dominant negative MEK proteins can suppress the tumourigenicity of ras transformed cell lines (Mansour, S. J., et al., Science, 265, 966-970 (1994); Arboleda et al., Methods Enzymol. (2001); 332: 353-67, Cowely, S., et al., Cell, 77, 841-852 (1994)). Expression of a dominant negative raf protein has also been found to inhibit ras dependent signalling as has suppression of raf expression using an antisense oligonucleotide construct (Koch, W., et al., Nature, 349, 426-428 (1991); Bruder, T. T., et al., Genes and Development, 6, 545-556 (1992))

Therefore evidence suggests that inhibition of raf kinase activity could be beneficial in the treatment of cancer and that targeting inhibition of B-raf could be particularly beneficial in those cancers containing a constitutively activated B-raf mutation.

The raf-MEK-ERK pathway functions downstream of many receptors and stimuli indicating a broad role in regulation of cell function. For this reason inhibitors of raf may find utility in other disease conditions which are associated with upregulation of signalling via this pathway. The raf-MEK-ERK pathway is also an important component of the normal response of non-transformed cells to growth factor action. Therefore inhibitors of raf may be of use in diseases where there is inappropriate or excessive proliferation of normal tissues. These include, but are not limited to glomerulonephritis and psoriasis.

The raf-MEK-ERK pathway is important in the action of growth factors that maintain host derived blood vessels supplying the tumours and in the initiation of new vessel formation as tumours grow and new tumours form from metastasising tumour cells. Growth factors that act in such a way on the blood vessels include the vascular endothelial growth factor family (VEGF) particularly those factors acting via VEGF receptor type 2, Tie-2 family, Ephrin growth factors. Inhibition of raf signalling will prevent the action of these growth factors and as a consequence limit growth of new tumour associated blood vessels and act to destroy the existing blood vessels associated with the tumour.

Cancer

Cancer is the collective term given to a group of diseases characterised by abnormal and uncontrolled cell growth. Normally, cells grow and divide to form new cells only when the body needs them. When cells grow old and die, new cells take their place. Mutations in the genes within a cell can sometimes disrupt this process such that new cells form when the body does not need them, and old cells do not die when they should. The extra cells form a mass of tissue, called a growth, neoplasm, or tumour. Tumours can be either benign (not cancerous) or malignant (cancerous). Benign tumors do not spread to other parts of the body, and they are rarely a threat to life whereas malignant tumors can spread (metastasize) and may be life threatening. Cancers originate within a single cell and hence can be classified by the type of cell in which they originate and by the location of the cell. Thus, Adenomas originate from glandular tissue, Carcinomas originate in epithelial cells, Leukaemias start in the bone marrow stem cells, Lymphomas originate in lymphatic tissue, Melanomas arise in melanocytes, Sarcomas begin in the connective tissue of bone or muscle and Teratomas begin within germ cells.

Various methods exist for treating cancers and the commonest are surgery, chemotherapy and radiation therapy. In general the choice of therapy will depend upon the location and grade of the tumour and the stage of the disease. If the tumour is localized, surgery is often the preferred treatment. Examples of common surgical procedures include prostatectomy for prostate cancer and mastectomy for breast cancer. The goal of the surgery can be either the removal of only the tumour, or the entire organ. Since a single cancer cell can grow into a sizeable tumor, removing only the tumour leads to a greater chance of recurrence. Chemotherapy involves the treatment of cancer with drugs that can destroy or prevent the growth of cancer cells. Alternative mechanisms exploited by cancer chemotherapies include anti-angiogenic agents which act to disrupt the blood vessels supplying the tumour and immunotherapeutic agents which act to enhance the host immune response against the tumour tissue. Normal cells grow and die in a controlled way. When cancer occurs, cells in the body that are not normal keep dividing and forming more cells without control. One class of anticancer drugs acts by killing dividing cells or by stopping them from growing or multiplying. Healthy cells can also be harmed, especially those that divide quickly, and this can lead to side effects. Radiation therapy involves the use of ionizing radiation to kill cancer cells and shrink tumours. Radiation therapy injures or destroys cells in the area being treated (the “target tissue”) by damaging their genetic material, making it impossible for these cells to continue to grow and divide. Although radiation damages both cancer cells and normal cells, most normal cells can recover from the effects of radiation and function properly. The goal of radiation therapy is to damage as many cancer cells as possible, while limiting harm to nearby healthy tissue. Radiation therapy may be used to treat almost every type of solid tumor, including cancers of the brain, breast, cervix, larynx, lung, pancreas, prostate, skin, spine, stomach, uterus, or soft tissue sarcomas. Radiation can also be used to treat leukaemia and lymphoma (cancers of the blood-forming cells and lymphatic system, respectively).

A large number of compounds of very diverse structure and biological properties have been used or proposed for use in the treatment of cancers, and some examples of these are set out in the section below headed “Prior Art”.

Prevention of Angiogenesis

Chronic proliferative diseases such as cancer are often accompanied by profound angiogenesis, which can contribute to or maintain an inflammatory and/or proliferative state, or which leads to tissue destruction through the invasive proliferation of blood vessels. (Folkman, EXS, 79, 1-81 (1997); Folkman, Nature Medicine, 1, 27-31 (1995); Folkman and Shing, J. Biol. Chem., 267, 10931 (1992)).

Angiogenesis is generally used to describe the development of new or replacement blood vessels, or neovascularisation. It is a necessary and physiological normal process by which the vasculature is established in the embryo. Angiogenesis does not occur, in general, in most normal adult tissues, exceptions being sites of ovulation, menses and wound healing. Many diseases, however, are characterized by persistent and unregulated angiogenesis. For instance, in arthritis, new capillary blood vessels invade the joint and destroy cartilage (Colville-Nash and Scott, Ann. Rhum, Dis., 51, 919 (1992)). In diabetes (and in many different eye diseases), new vessels invade the macula or retina or other ocular structures, and may cause blindness (Brooks, et al., Cell, 79, 1157 (1994)). The process of atherosclerosis has been linked to angiogenesis (Kahlon, et al., Can. J. Cardiol., 8, 60 (1992)). Tumor growth and metastasis have been found to be angiogenesis-dependent (Folkman, Cancer Biol, 3, 65 (1992); Denekamp, Br. J. Rad., 66,181 (1993); Fidler and Ellis, Cell, 79,185 (1994)).

The recognition of the involvement of angiogenesis in major diseases has been accompanied by research to identify and develop inhibitors of angiogenesis. These inhibitors are generally classified in response to discrete targets in the angiogenesis cascade, such as activation of endothelial cells by an angiogenic signal; synthesis and release of degradative enzymes; endothelial cell migration; proliferation of endothelial cells; and formation of capillary tubules. Therefore, angiogenesis occurs in many stages and attempts are underway to discover and develop compounds that work to block angiogenesis at these various stages.

There are publications that teach that inhibitors of angiogenesis, working by diverse mechanisms, are beneficial in diseases such as cancer and metastasis (O'Reilly, et al., Cell, 79, 315 (1994); Ingber, et al., Nature, 348, 555 (1990)), ocular diseases (Friedlander, et al., Science, 270,1500 (1995)), arthritis (Peacock, et al., J. Exp. Med., 175, 1135 (1992); Peacock et al., Cell. Immun., 160,178 (1995)) and hemangioma (Taraboletti, et al., J. Natl. Cancer Inst., 87, 293 (1995)).

RTKs

Receptor tyrosine kinases (RTKs) are important in the transmission of biochemical signals across the plasma membrane of cells. These transmembrane molecules characteristically consist of an extracellular ligand-binding domain connected through a segment in the plasma membrane to an intracellular tyrosine kinase domain. Binding of ligand to the receptor results in stimulation of the receptor-associated tyrosine kinase activity that leads to phosphorylation of tyrosine residues on both the receptor and other intracellular proteins, leading to a variety of cellular responses. To date, at least nineteen distinct RTK subfamilies, defined by amino acid sequence homology, have been identified.

FGFR

The fibroblast growth factor (FGF) family of signalling polypeptides regulates a diverse array of physiologic functions including mitogenesis, wound healing, cell differentiation and angiogenesis, and development. Both normal and malignant cell growth as well as proliferation are affected by changes in local concentration of these extracellular signalling molecules, which act as autocrine as well as paracrine factors. Autocrine FGF signalling may be particularly important in the progression of steroid hormone-dependent cancers and to a hormone independentstate (Powers, et al., Endocr. Relat. Cancer, 7, 165-197 (2000)).

FGFs and their receptors are expressed at increased levels in several tissues and cell lines and overexpression is believed to contribute to the malignant phenotype. Furthermore, a number of oncogenes are homologues of genes encoding growth factor receptors, and there is a potential for aberrant activation of FGF-dependent signalling in human pancreatic cancer (Ozawa, et al., Teratog. Carcinog. Mutagen., 21, 27-44 (2001)).

The two prototypic members are acidic fibroblast growth factor (aFGF or FGF1) and basic fibroblast growth factors (bFGF or FGF2), and to date, at least twenty distinct FGF family members have been identified. The cellular response to FGFs is transmitted via four types of high affinity transmembrane tyrosine-kinase fibroblast growth factor receptors numbered 1 to 4 (FGFR-1 to FGFR-4). Upon ligand binding, the receptors dimerize and auto-or trans-phosphorylate specific cytoplasmic tyrosine residues to transmit an intracellular signal that ultimately reaches nuclear transcription factor effectors.

Disruption of the FGFR-1 pathway should affect tumor cell proliferation since this kinase is activated in many tumor types in addition to proliferating endothelial cells. The over-expression and activation of FGFR-1 in tumor-associated vasculature has suggested a role for these molecules in tumor angiogenesis.

Fibroblast growth factor receptor 2 has high affinity for the acidic and/or basic fibroblast growth factors, as well as the keratinocyte growth factor ligands. Fibroblast growth factor receptor 2 also propagates the potent osteogenic effects of FGFs during osteoblast growth and differentiation. Mutations in fibroblast growth factor receptor 2, leading to complex functional alterations, were shown to induce abnormal ossification of cranial sutures (craniosynostosis), implying a major role of FGFR signalling in intramembranous bone formation. For example, in Apert (AP) syndrome, characterized by premature cranial suture ossification, most cases are associated with point mutations engendering gain-of-function in fibroblast growth factor receptor 2 (Lemonnier, et al., J. Bone Miner. Res., 16, 832-845 (2001)).

Several severe abnormalities in human skeletal development, including Apert, Crouzon, Jackson-Weiss, Beare-Stevenson cutis gyrata, and Pfeiffer syndromes are associated with the occurrence of mutations in fibroblast growth factor receptor 2. Most, if not all, cases of Pfeiffer Syndrome (PS) are also caused by de novo mutation of the fibroblast growth factor receptor 2 gene (Meyers, et al., Am. J. Hum. Genet., 58, 491-498 (1996); Plomp, et al., Am. J. Med. Genet., 75, 245-251 (1998)), and it was recently shown that mutations in fibroblast growth factor receptor 2 break one of the cardinal rules governing ligand specificity. Namely, two mutant splice forms of fibroblast growth factor receptor, FGFR2c and FGFR2b, have acquired the ability to bind to and be activated by atypical FGF ligands. This loss of ligand specificity leads to aberrant signalling and suggests that the severephenotypes of these disease syndromes result from ectopic ligand-dependent activation of fibroblast growth factor receptor 2 (Yu, et al., Proc. Natl. Acad. Sci. U.S.A., 97, 14536-14541 (2000)).

Activating mutations of the FGF-R3 receptor tyrosine kinase such as chromosomal translocations or point mutations produce deregulated, constitutively active, FGF-R3 receptors which have been involved in multiple myeloma and in bladder and cervix carcinomas (Powers, C. J., et al., Endocr. Rel. Cancer, 7, 165 (2000)). Accordingly, FGFR-3 inhibition would be useful in the treatment of multiple myeloma, bladder and cervix carcinomas.

VEGFR

Vascular endothelial growth factor (VEGF), a polypeptide, is mitogenic for endothelial cells in vitro and stimulates angiogenic responses in vivo. VEGF has also been linked to inappropriate angiogenesis (Pinedo, H. M., et al., The Oncologist, 5(90001), 1-2 (2000)). VEGFR(s) are protein tyrosine kinases (PTKs). PTKs catalyze the phosphorylation of specific tyrosyl residues in proteins involved in the regulation of cell growth and differentiation. (Wilks, A. F., Progress in Growth Factor Research, 2, 97-111 (1990); Courtneidge, S. A., Dev. Supp.l, 57-64 (1993); Cooper, J. A., Semin. Cell Biol., 5(6), 377-387 (1994); Paulson, R. F., Semin. Immunol., 7(4), 267-277 (1995); Chan, A. C., Curr. Opin. Immunol, 8(3), 394-401 (1996)).

Three PTK receptors for VEGF have been identified: VEGFR-1 (Flt-1); VEGFR-2 (Flk-1 or KDR) and VEGFR-3 (Flt-4). These receptors are involved in angiogenesis and participate in signal transduction (Mustonen, T., et al., J. Cell Biol., 129, 895-898 (1995)).

Of particular interest is VEGFR-2, which is a transmembrane receptor PTK expressed primarily in endothelial cells. Activation of VEGFR-2 by VEGF is a critical step in the signal transduction pathway that initiates tumour angiogenesis. VEGF expression may be constitutive to tumour cells and can also be upregulated in response to certain stimuli. One such stimuli is hypoxia, where VEGF expression is upregulated in both tumour and associated host tissues. The VEGF ligand activates VEGFR-2 by binding with its extracellular VEGF binding site. This leads to receptor dimerization of VEGFRs and autophosphorylation of tyrosine residues at the intracellular kinase domain of VEGFR-2. The kinase domain operates to transfer a phosphate from ATP to the tyrosine residues, thus providing binding sites for signalling proteins downstream of VEGFR-2 leading ultimately to initiation of angiogenesis (McMalion, G., The Oncologist, 5(90001), 3-10 (2000)).

Inhibition at the kinase domain binding site of VEGFR-2 would block phosphorylation of tyrosine residues and serve to disrupt initiation of angiogenesis.

TIE

Angiopoieten 1 (Ang1), a ligand for the endothelium-specific receptor tyrosine kinase TIE-2 is a novel angiogenic factor (Davis, et al., Cell, 87, 1161-1169 (1996); Partanen, et al., Mol. Cell Biol., 12, 1698-1707 (1992); U.S. Pat. Nos. 5,521,073; 5,879,672; 5,877,020; and 6,030,831). The acronym TIE represents “tyrosine kinase containing Ig and EGF homology domains”. TIE is used to identify a class of receptor tyrosine kinases, which are exclusively expressed in vascular endothelial cells and early hemopoietic cells. Typically, TIE receptor kinases are characterized by the presence of an EGF-like domain and an immunoglobulin (IG) like domain, which consists of extracellular folding units, stabilized by intra-chain disulfide bonds (Partanen, et al., Curr. Topics Microbiol. Immunol., 237, 159-172 (1999)). Unlike VEGF, which functions during the early stages of vascular development, Ang1 and its receptor TIE-2 function in the later stages of vascular development, i.e. during vascular remodelling (remodelling refers to formation of a vascular lumen) and maturation (Yancopoulos, et al., Cell, 93, 661-664 (1998); Peters, K. G., Circ. Res., 83(3), 342-3 (1998); Suri, et al., Cell, 87, 1171-1180 (1996)).

Consequently, inhibition of TIE-2 would be expected to serve to disrupt remodelling and maturation of new vasculature initiated by angiogenesis thereby disrupting the angiogenic process.

Eph

The largest subfamily of receptor tyrosine kinases (RTKs), the Eph family, and their ligands (ephrins), play important roles in physiologic and pathologic vascular processes. Both the Ephs (receptors) and ephrins (ligands) are divided into two groups, A and B subfamilies (Eph Nomenclature Committee, 1997). The binding of ephrin ligands to Eph receptors is dependent on cell-cell interactions. The interactions of ephrins and Ephs have recently been shown to function via bi-directional signalling. The ephrins binding to Eph receptors initiate phosphorylation at specific tyrosine residues in the cytoplasmic domain of the Eph receptors. In response to Eph receptor binding, the ephrin ligand also undergoes tyrosine phosphorylation, so-called ‘reverse’ signalling (Holland, S. J., et al., Nature, 383, 722-725 (1996); Bruckner et al., Science 275: 1640-1643 (1997)).

Eph RTKs and their ephrin ligands play important roles in embryonic vascular development. Disruption of specific Eph receptors and ligands (including ephrin-B2) leads to defective vessel remodelling, organisation, and sprouting resulting in embryonic death (Wang, H. U., et al., Cell, 93: 741-753 (1998); Adams, R. H., et al., Genes Dev, 13, 295-306 (1999); Gale and Yancopoulos, Genes Dev, 13, 1055-1066 (1999); Helbling, P. M., et al., Development, 127, 269-278 (2000)). Coordinated expression of the Eph/ephrin system determines the phenotype of embryonic vascular structures: ephrin-B2 is present on arterial endothelial cells (ECs), whereas EphB4 is present on venous ECs (Gale and Yancopoulos, Genes Dev, 13, 1055-1066 (1999); Shin, D., et al., Dev Biol, 230, 139-150 (2001)). Recently, specific Ephs and ephrins have been implicated in tumour growth and angiogenesis.

The Ephs and ephrins have been found to be overexpressed in many human tumours. In particular, the role of EphB2 has been identified in small cell lung carcinoma (Tang, X. X., et al., Clin Cancer Res, 5, 455-460 (1999)), human neuroblastomas (Tang, X. X., et al., Clin Cancer Res, 5, 1491-1496 (1999)) and colorectal cancers (Liu, W., et al., Brit. J. Canc., 90, 1620-1626 (2004)), and higher expression levels of Ephs and ephrins, including EphB2, have been found to correlate with more aggressive and metastatic tumours (Nakamoto, M. and Bergemann, A. D., Microsc. Res. Tech, 59, 58-67 (2002)).

Consequently, inhibition of EphB2 would be expected to serve to disrupt angiogenesis, and in particular in certain tumours where over-expression occurs.

Prior Art

U.S. Pat. No. 6,414,013 (Pharmacia & Upjohn) discloses 3-aminocarbonyl-2-carboxamidothiophenes that have activity as kinase inhibitors and which are considered to be useful in the treatment of a variety of diseases including cancers, arthritis and autoimmune diseases.

U.S. Pat. No. 4,767,758 (CNDR) describes thiophene analogues that are useful in treating tumours. The thiophenes can contain amide substituents.

WO 00/71535 (Scios Inc.) discloses indole-type compounds as inhibitors of p38 kinase.

WO 93/1408 (Smith-Kline Beecham) discloses 1,3,4-triaryl imidazoles as inhibitors of p38 MAP kinase.

WO 99/15164 (Zeneca) discloses various bis-benzamidophenyl derivatives compounds which exhibit inhibition of p38 activity.

WO 99/00357 (Vertex) discloses a further class of diarylurea compounds as p38 MAP kinase inhibitors.

WO 03/004020 (Boehringer Ingelheim) discloses a class of heteroaryl diamides in which one amide group contains a phenyl, pyridyl or pyrimidinyl group having a carbocyclic or heterocyclic group bonded to the ortho position thereof either directly or through an intervening linker atom or group. The compounds are described as being inhibitors of the microsomal triglyceride transfer protein and therefore useful in lowering plasma lipoprotein levels.

WO 96/41795 (Fujisawa) discloses thiophene diamides that are useful as vasopressin antagonists.

WO 94/04525 (Otsuka) discloses benzazepines and aza analogues in which a nitrogen atom of the benzazepine group is attached to an amide group that can contain a heterocyclic ring such as a thiophene. The compounds are vasopressin and oxytocin antagonists.

EP 0 592 167 (Zeneca) describes antibiotic thiopenem derivatives containing an optionally N-substituted pyrrolidine ring that can be linked via an amide bond to a thiophene group.

A. Khalaf et al. Tetrahedron, (2000), 56 (29), 5225-5239 describes a thiophene diamide containing a 5-nitro-2-thiophenyl group. The compound is stated to be a DNA minor groove binder.

JP 10212271 (Zeria) (Chem. Abstract 129:202763) describes a class of compounds that are useful in the treatment of digestive tract disorders. The compounds are amides that can contain a thiophene carboxylic acid amide group. Also disclosed as intermediates are the corresponding carboxylic acid esters.

JP 05230009 (Taisho) discloses as inhibitors of Platelet-Activating Factor (PAF) compounds, N-substituted amides of 5-(4-carbamimidoyl-benzoylamino)-thiophene-2-carboxylic acid. The amide N-substituent groups contain an alkylene chain terminating in a carboxylic acid or alkoxycarbonyl group.

WO 01/40223 discloses a class of pesticidal substituted aminoheterocyclylamides.

Gewald et al., J für Prakt. Chem., (Leipzig), (1991), 333(2), 229-36 describes the reactions of 2-aminiothiophene-3-carbonitriles with heterocumulenes. The article discloses a urea, each nitrogen atom of which bears a 2-ethoxycarbonyl-3-methyl-4-cyanothien-2-yl group.

U.S. Pat. No. 5,571,810 (Fujisawa) describes 2,3-diaryl thiophenes that have anti-inflammatory and analgesic activity and which are considered to be useful in treating a range of diseases including rheumatoid arthritis.

WO 99/32455 (Bayer) describes a series of phenyl imidazolyl ureas that act as raf kinase inhibitors.

WO 01/98301 (Japan Tobacco) discloses a class of pyrazolopyridine compounds that prevent or inhibit fibrosis.

WO98/52558 (Bayer Corporation) describes a class of aryl ureas as p38 MAP kinase inhibitors. The aryl ureas can contain a thiophene unit.

EP 1253142 discloses various heteroaryl compounds as thrombopoietin receptor agonists.

WO 01/40223 discloses a class of pesticidal substituted aminoheterocyclylamides.

An article by A. R. Redman et al., in Bioorganic & Medicinal Chemistry Letters, 11, 9-12, (2001) describes thienyl compounds, in particular thienyl ureas, having p38 kinase inhibitory activity. The compounds disclosed in Redman et al. are characterised by the presence of an aryl ureido group at the 3-position of the thiophene ring.

WO 99/32111 and WO 99/32463 (both to Bayer Corporation) each disclose a class of diaryl/heteroaryl urea compounds as MAP kinase inhibitors. The compounds can contain a 5-substituted thiophen-2-yl group but there is no disclosure of compounds containing a morpholino-methyl group.

WO 99/32106 (Bayer Corporation) discloses a class of compounds of similar structure to those of WO 99/32111 for use as raf kinase inhibitors.

WO 99/32477 (Schering) discloses heterocyclic amide derivatives as anti-coagulants.

EP 0716855 & WO 95/10513 (both to Pfizer) each describe benzthiophene compounds that have estrogen agonist activity.

### SUMMARY OF THE INVENTION

The present invention provides a further class of compounds that have p38 MAP kinase inhibiting or modulating activity, and which it is envisaged will be useful in preventing or treating disease states or conditions mediated by p38 MAP kinase.

The invention also provides compounds that inhibit raf kinases and compounds which it is envisaged will be useful for the treatment or prophylaxis of cancers and for inhibiting or preventing undesirable angiogenesis.

Accordingly, in a first aspect, the invention provides a compound of the formula (I): 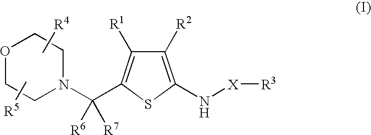
or a salt, solvate or N-oxide thereof, wherein:
R¹ and R² are the same or different and each is selected from hydrogen, saturated C₁₋₃ hydrocarbyl, halogen and cyano;
X is selected from C═O, C═S, C(═O)NH, C(═S)NH, C(═O)O, C(═O)S, C(═S)O and C(═S)S;
R³ is selected from aryl and heteroaryl groups each having from 5 to 12 ring members and being unsubstituted or substituted by one or more substituent groups R¹⁰;
R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR_{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; or two adjacent groups R¹⁰, together with the carbon atoms or heteroatoms to which they are attached may form a 5-membered heteroaryl ring or a 5- or 6-membered non-aromatic heterocyclic ring, wherein the said heteroaryl and heterocyclic groups contain up to 3 heteroatom ring members selected from N, O and S;
R^{c} is selected from hydrogen and C₁₋₄ hydrocarbyl; and
X¹ is O, S or NR^{c} and X² is ═O, ═S or ═NR^{c};
R⁴ and R⁵ are the same or different and are selected from hydrogen and methyl; or one of R⁴ and R⁵ is selected from hydroxymethyl and ethyl and the other is hydrogen; and
R⁶ and R⁷ are the same or different and are selected from hydrogen and methyl.

The invention further provides:
A method for the prophylaxis or treatment of a disease state or condition of the type defined herein, which method comprises administering to a subject (e.g. a human subject) in need thereof a compound of the formula (I), (II), (III), (IV), (V) or (VI) or any sub-group thereof as defined herein.
A compound of the formula (I), (II), (III), (IV), (V) or (VI) or any sub-group thereof as defined herein for use in the prophylaxis or treatment of a disease state or condition mediated by a p38 MAP kinase.
The use of a compound of the formula (I), (II), (III), (IV), (V) or (VI) or any sub-group thereof as defined herein for the manufacture of a medicament for the prophylaxis or treatment of a disease state or condition mediated by a p38 MAP kinase.
A method for the prophylaxis or treatment of a disease state or condition mediated by a p38 MAP kinase, which method comprises administering to a subject (e.g. a human subject) in need thereof a compound of the formula (I), (II), (III), (IV), (V) or (VI) or any sub-group thereof as defined herein.
A method of inhibiting a p38 MAP kinase, which method comprises contacting the p38 MAP kinase with a kinase-inhibiting compound of the formula (I), (II), (III), (IV), (V) or (VI) or any sub-group thereof as defined herein.
A method of modulating a cellular process by inhibiting the activity of a p38 MAP kinase using a compound of the formula (I) as defined herein, which method comprises bringing the compound of formula (I), (II), (III), (IV), (V) or (VI) or any sub-group thereof into contact with a cellular environment containing the p38 MAP kinase.

In a further aspect, the invention provides a compound of the formula (I), (II), (III), (IV), (V) or (VI) or any sub-group thereof for use in medicine, for example for use in therapy.

In another aspect, the invention provides a pharmaceutical composition comprising a compound of the formula (I), (II), (III), (IV), (V) or (VI) or any sub-group thereof in association with a pharmaceutically acceptable carrier.

In further aspects, the invention also provides:
The use of a compound of the formula (I), (II), (III), (IV), (V) or (VI) or any sub-group thereof as defined herein for the manufacture of a medicament for the prophylaxis or treatment of a cancer.
A compound of the formula (I), (II), (III), (IV), (V) or (VI) or any sub-group thereof as defined herein for use in the treatment or prophylaxis of a cancer.
A method for treating a disease or condition comprising or arising from abnormal cell growth in a mammal, the method comprising administering to the mammal a therapeutically effective amount of a compound of the formula (I), (II), (III), (IV), (V) or (VI) or any sub-group thereof as defined herein.
The use of a compound of the formula (I), (II), (III), (IV), (V) or (VI) or any sub-group thereof as defined herein for the manufacture of a medicament for the prophylaxis or treatment of a disease state or condition arising from abnormal cell growth.
A method for treating a disease or condition comprising or arising from abnormal cell growth in a mammal, which method comprises administering to the mammal a compound of the formula (I), (II), (III), (IV), (V) or (VI) or any sub-group thereof as defined herein in an amount effective in inhibiting abnormal cell growth.
A method for alleviating or reducing the incidence of a disease or condition comprising or arising from abnormal cell growth in a mammal, which method comprises administering to the mammal a compound of the formula (I), (II), (III), (IV), (V) or (VI) or any sub-group thereof as defined herein in an amount effective in inhibiting abnormal cell growth.
A method for alleviating or reducing the incidence of a disease state or condition disclosed herein, which method comprises administering to a patient (e.g. a patient in need thereof) a compound (e.g. a therapeutically effective amount) of the formula (I), (II), (III), (IV), (V) or (VI) or any sub-group thereof as defined herein.
A compound of the formula (I), (II), (III), (IV), (V) or (VI) or any sub-group thereof as defined herein for use in the prophylaxis or treatment of a disease state or condition mediated by a raf kinase (such as B-raf or C-raf).
The use of a compound of formula (I), (II), (III), (IV), (V) or (VI) or any sub-group thereof as defined herein for the manufacture of a medicament for the prophylaxis or treatment of a disease state or condition mediated by a raf kinase (such as B-raf or C-raf).
A method for the prophylaxis or treatment of a disease state or condition mediated by a raf kinase (such as B-raf or C-raf)., which method comprises administering to a subject in need thereof a compound of the formula (I), (II), (III), (IV), (V) or (VI) or any sub-group thereof as defined herein.
A method for treating a disease or condition comprising or arising from abnormal cell growth in a mammal, the method comprising administering to the mammal a compound of the formula (I), (II), (III), (IV), (V) or (VI) or any sub-group thereof as defined herein in an amount effective to inhibit raf kinase (such as B-raf or C-raf) activity.
A method of inhibiting a raf kinase (such as B-raf or C-raf), which method comprises contacting the kinase with a kinase-inhibiting compound of the formula (I), (II), (III), (IV), (V) or (VI) or any sub-group thereof as defined herein.
A method of modulating a cellular process (for example proliferation or cell division) by inhibiting the activity of a raf kinase (such as B-raf or C-raf) using a compound of the formula (I), (II), (III), (IV), (V) or (VI) or any sub-group thereof as defined herein.
A method for the diagnosis and treatment of a disease state or condition mediated by a raf kinase (such as B-raf or C-raf), which method comprises (i) screening a patient to determine whether a disease or condition from which the patient is or may be suffering is one which would be susceptible to treatment with a compound having activity against a raf kinase (such as B-raf or C-raf); and (ii) where it is indicated that the disease or condition from which the patient is thus susceptible, thereafter administering to the patient a compound of the formula (I), (II), (III), (IV), (V) or (VI) or any sub-group thereof as defined herein.
The use of a compound of the formula (I), (II), (III), (IV), (V) or (VI) or any sub-group thereof as defined herein for the manufacture of a medicament for the treatment or prophylaxis of a disease state or condition in a patient who has been screened and has been determined as suffering from, or being at risk of suffering from, a disease or condition which would be susceptible to treatment with a compound having activity against a raf kinase (such as B-raf or C-raf).
A compound of the formula (I), (II), (III), (IV), (V) or (VI) or any sub-group thereof as defined herein for use in the prophylaxis or treatment of inappropriate, excessive or undesirable angiogenesis.
The use of a compound of the formula (I), (II), (III), (IV), (V) or (VI) or any sub-group thereof as defined herein for the manufacture of a medicament for the prophylaxis or treatment of inappropriate, excessive or undesirable angiogenesis.
A compound of the formula (I), (II), (III), (IV), (V) or (VI) or any sub-group thereof as defined herein for use in the prophylaxis or treatment or alleviation of diseases or conditions, characterised by the up-regulation of a receptor tyrosine kinase, and in particular FGFR, Tie, VEGFR and/or Eph (more particularly a tyrosine kinase selected from FGFR-1, FGFR-2, FGFR-3, Tie2, VEGFR-2 and EphB2).
The use of a compound of the formula (I), (II), (III), (IV), (V) or (VI) or any sub-group thereof as defined herein for the manufacture of a medicament for the prophylaxis or treatment or alleviation of diseases or conditions, characterised by the up-regulation of a receptor tyrosine kinase, and in particular FGFR, Tie, VEGFR and/or Eph (more particularly a tyrosine kinase selected from FGFR-1, FGFR-2, FGFR-3, Tie2, VEGFR-2 and EphB2).
A method of inhibiting angiogenesis in vitro or in vivo, comprising contacting a cell with an effective amount of a compound of the formula (I), (II), (III), (IV), (V) or (VI) or any sub-group thereof as defined herein.
A method for the treatment or alleviation of inappropriate, excessive or undesirable angiogenesis comprising administering to a subject suffering from said a disease or condition ameliorated by the inhibition of angiogenesis a therapeutically-effective amount of a compound of the formula (I), (II), (III), (IV), (V) or (VI) or any sub-group thereof as defined herein.
A method for the treatment of a disease or condition, preferably cancer, characterised by the up-regulation of a receptor tyrosine kinase comprising:
   (i) diagnosing a subject suffering from a disease or condition, preferably cancer, characterised by the up-regulation or activating mutants of a receptor tyrosine kinase (for example a receptor tyrosine kinase selected from FGFR, Tie, VEGFR and Eph, and more particularly from FGFR-1, FGFR-2, FGFR-3, Tie2, VEGFR-2 and EphB2); and
   (ii) administering to said subject a therapeutically-effective amount of a compound of the formula (I), (II), (III), (IV), (V) or (VI) or any sub-group thereof as defined herein.
A method for the treatment of diseases, for example cancers, with:
   (a) activating mutants of ras or raf;
   (b) upregulation of ras or raf;
   (c) upregulated raf-MEK-ERK pathway signals; or
   (d) upregulation of growth factor receptors, such as ERB2 and EGFR, comprising:
   (i) diagnosing a subject suffering from a disease with:
   (a) activating mutants of ras or raf;
   (b) upregulation of ras or raf;
   (c) upregulated raf-MEK-ERK pathway signals; or
   (d) upregulation of growth factor receptors, such as ERB2 and EGFR;
(ii) administering to said subject a therapeutically-effective amount of a raf kinase inhibitor compound of the formula (I), (II), (III), (IV), (V) or (VI) or any sub-group thereof as defined herein.
   General Preferences and Definitions

In this specification, references to formula (I) include any sub-group (e.g. formulae (II), (III), (IV), (V) or (VI)), example or embodiment of formula (I), unless the context indicates otherwise. Thus for example, references to inter alia therapeutic uses, pharmaceutical formulations and processes for making compounds, where they refer to formula (I), are also to be taken as referring to any other sub-group of compounds or embodiment of formula (I). Similarly, where preferences, embodiments and examples are given for compounds of the formula (I), they are also applicable to any sub-groups or embodiments of formula (I) unless the context requires otherwise.

As used herein, references to the “upregulation of a kinase” include elevated expression or over-expression of the kinase, including gene amplification (i.e. multiple gene copies) and increased expression by a transcriptional effect, and hyperactivity and activation of the kinase, including activation by mutations.

The following general preferences and definitions shall apply to each of the moieties R¹ to R⁷, R¹⁰, R^{a}, R^{b}, R^{c}, X, X¹ and X² and any sub-definition, sub-group or embodiment thereof, unless the context indicates otherwise.

References to “carbocyclic” and “heterocyclic” groups as used herein, either in the context of R³ and sub-definitions thereof or otherwise, shall, unless the context indicates otherwise, include both aromatic and non-aromatic ring systems. In general, such groups may be monocyclic or bicyclic and may contain, for example, 3 to 12 ring members, more usually 5 to 10 ring members. Examples of monocyclic groups are groups containing 3, 4, 5, 6, 7, and 8 ring members, more usually 3 to 7, and preferably 5 or 6 ring members. Examples of bicyclic groups are those containing 8, 9, 10, 11 and 12 ring members, and more usually 9 or 10 ring members.

The carbocyclic or heterocyclic groups can be aryl or heteroaryl groups having from 5 to 12 ring members, more usually from 5 to 10 ring members. The term “aryl” as used herein refers to a carbocyclic group having aromatic character and the term “heteroaryl” is used herein to denote a heterocyclic group having aromatic character. The terms “aryl” and “heteroaryl” embrace polycyclic (e.g. bicyclic) ring systems wherein one or more rings are non-aromatic, provided that at least one ring is aromatic. In such polycyclic systems, the group may be attached by the aromatic ring, or by a non-aromatic ring. The aryl or heteroaryl groups can be monocyclic or bicyclic groups and can be unsubstituted or substituted with one or more substituents, for example one or more groups R¹⁰ as defined herein.

The term non-aromatic group embraces unsaturated ring systems without aromatic character, partially saturated and fully saturated carbocyclic and heterocyclic ring systems. The terms “unsaturated” and “partially saturated” refer to rings wherein the ring structure(s) contains atoms sharing more than one valence bond i.e. the ring contains at least one multiple bond e.g. a C═C, C—C or N═C bond. The term “fully saturated” refers to rings where there are no multiple bonds between ring atoms. Saturated carbocyclic groups include cycloalkyl groups as defined below. Partially saturated carbocyclic groups include cycloalkenyl groups as defined below, for example cyclopentenyl, cycloheptenyl and cyclooctenyl.

Examples of heteroaryl groups are monocyclic and bicyclic groups containing from five to twelve ring members, and more usually from five to ten ring members. The heteroaryl group can be, for example, a five membered or six membered monocyclic ring or a bicyclic structure formed from fused five and six membered rings or two fused six membered rings. Each ring may contain up to about four heteroatoms typically selected from nitrogen, sulphur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of five membered heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups.

Examples of six membered heteroaryl groups include but are not limited to pyridine, pyrazine, pyridazine, pyrimidine and triazine.

A bicyclic heteroaryl group may be, for example, a group selected from:
a) a benzene ring fused to a 5- or 6-membered ring containing 1, 2 or 3 ring heteroatoms;
b) a pyridine ring fused to a 5- or 6-membered ring containing 1, 2 or 3 ring heteroatoms;
c) a pyrimidine ring fused to a 5- or 6-membered ring containing 1 or 2 ring heteroatoms;
d) a pyrrole ring fused to a a 5- or 6-membered ring containing 1, 2 or 3 ring heteroatoms;
e) a pyrazole ring fused to a a 5- or 6-membered ring containing 1 or 2 ring heteroatoms;
f) an imidazole ring fused to a 5- or 6-membered ring containing 1 or 2 ring heteroatoms;
g) an oxazole ring fused to a 5- or 6-membered ring containing 1 or 2 ring heteroatoms;
h) an isoxazole ring fused to a 5- or 6-membered ring containing 1 or 2 ring heteroatoms;
i) a thiazole ring fused to a 5- or 6-membered ring containing 1 or 2 ring heteroatoms;
j) an isothiazole ring fused to a 5- or 6-membered ring containing 1 or 2 ring heteroatoms;
k) a thiophene ring fused to a 5- or 6-membered ring containing 1, 2 or 3 ring heteroatoms;
l) a furan ring fused to a 5- or 6-membered ring containing 1, 2 or 3 ring heteroatoms;
m) an oxazole ring fused to a 5- or 6-membered ring containing 1 or 2 ring heteroatoms;
n) an isoxazole ring fused to a 5- or 6-membered ring containing 1 or 2 ring heteroatoms;
o) a cyclohexyl ring fused to a 5- or 6-membered ring containing 1, 2 or 3 ring heteroatoms; and
p) a cyclopentyl ring fused to a 5- or 6-membered ring containing 1, 2 or 3 ring heteroatoms.

Particular examples of bicyclic heteroaryl groups containing a six membered ring fused to a five membered ring include but are not limited to benzfuran, benzthiophene, benzimidazole, benzoxazole, benzisoxazole, benzthiazole, benzisothiazole, isobenzofuran, indole, isoindole, indolizine, indoline, isoindoline, purine (e.g., adenine, guanine), indazole, benzodioxole, pyrazolopyridine, pyrazolopyrimidine, pyrrolopyridine, pyrrolopyrimidine and pyrazolopyridine groups.

Particular examples of bicyclic heteroaryl groups containing two fused six membered rings include but are not limited to quinoline, isoquinoline, chroman, thiochroman, chromene, isochromene, chroman, isochroman, benzodioxan, quinolizine, benzoxazine, benzodiazine, pyridopyridine, quinoxaline, quinazoline, cinnoline, phthalazine, naphthyridine and pteridine groups.

Examples of polycyclic aryl and heteroaryl groups containing an aromatic ring and a non-aromatic ring include tetrahydronaphthalene, tetrahydroisoquinoline, tetrahydroquinoline, dihydrobenzthiene, dihydrobenzfuran, 2,3-dihydro-benzo[1,4]dioxine, benzo[1,3]dioxole, 4,5,6,7-tetrahydrobenzofuran, indoline and indane groups.

Examples of carbocyclic aryl groups include phenyl, naphthyl, indenyl, and tetraliydronaphthyl groups.

Examples of non-aromatic heterocyclic groups include unsubstituted or substituted (by one or more groups R¹⁰) heterocyclic groups having from 3 to 12 ring members, typically 4 to 12 ring members, and more usually from 5 to 10 ring members. Such groups can be monocyclic or bicyclic, for example, and typically have from 1 to 5 heteroatom ring members (more usually 1,2, 3 or 4 heteroatom ring members) typically selected from nitrogen, oxygen and sulphur.

When sulphur is present, it may, where the nature of the adjacent atoms and groups permits, exist as —S—, —S(O)— or —S(O)₂—.

The heterocylic groups can contain, for example, cyclic ether moieties (e.g. as in tetrahydrofuran and dioxane), cyclic thioether moieties (e.g. as in tetrahydrothiophene and dithiane), cyclic amine moieties (e.g. as in pyrrolidine), cyclic amide moieties (e.g. as in pyrrolidone), cyclic urea moieties (e.g. as in imidazolidin-2-one), cyclic thiourea moieties, cyclic thioamides, cyclic thioesters, cyclic ester moieties (e.g. as in butyrolactone), cyclic sulphones (e.g. as in sulpholane and sulpholene), cyclic sulphoxides, cyclic sulphonamides and combinations thereof (e.g. morpholine and thiomorpholine and its S-oxide and S,S-dioxide).

In one sub-group of compounds, the heterocyclic group can contain, for example, cyclic ether moieties (e.g. as in tetrahydrofuran and dioxane), cyclic thioether moieties (e.g. as in tetrahydrothiophene and dithiane), cyclic amine moieties (e.g. as in pyrrolidine), cyclic sulphones (e.g. as in sulfolane and sulfolene)), cyclic sulphoxides, cyclic sulphonamides and combinations thereof (e.g. thiomorpholine).

Examples of monocyclic non-aromatic heterocyclic groups include 5-, 6-and 7-membered monocyclic heterocyclic groups. Particular examples include morpholine, thiomorpholine and its S-oxide and S,S-dioxide (particularly thiomorpholine), piperidine (e.g. 1-piperidinyl, 2-piperidinyl 3-piperidinyl and 4-piperidinyl), N-alkyl piperidines such as N-methyl piperidine, piperidone, pyrrolidine (e.g. 1-pyrrolidinyl, 2-pyrrolidinyl and 3-pyrrolidinyl), pyrrolidone, azetidine, pyran (2H-pyran or 4H-pyran), dihydrothiophene, dihydropyran, dihydrofuran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazone, piperazine, and N-alkyl piperazines such as N-methyl piperazine, N-ethyl piperazine and N-isopropylpiperazine. In general, preferred non-aromatic heterocyclic groups include piperidine, pyrrolidine, azetidine, morpholine, piperazine and N-alkyl piperazines.

One sub-group of non-aromatic heterocyclic groups consists of morpholine, piperidine (e.g, 1-piperidinyl, 2-piperidinyl 3-piperidinyl and 4-piperidinyl), pyrrolidine (e.g. 1-pyrrolidinyl, 2-pyrrolidinyl and 3-pyrrolidinyl), pyrrolidone, pyran (2H-pyran or 4H-pyran), dihydrothiophene, dihydropyran, dihydrofuran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazine, and N-alkyl piperazines such as N-methyl piperazine. Within this sub-group, particular non-aromatic heterocyclic groups include morpholine and N-alkyl piperazines.

Examples of non-aromatic carbocyclic groups include cycloalkane groups such as cyclohexyl and cyclopentyl, cycloalkenyl groups such as cyclopentenyl, cyclohexenyl, cycloheptenyl and cyclooctenyl, as well as cyclohexadienyl, cyclooctatetraene, tetrahydronaphthenyl and decalinyl.

Preferred non-aromatic carbocyclic groups are monocyclic rings and most preferably saturated monocyclic rings.

Typical examples are three, four, five and six membered saturated carbocyclic rings, e.g. optionally substituted cyclopentyl and cyclohexyl rings.

One sub-set of non-aromatic carbocyclic groups includes unsubstituted or substituted (by one or more groups R¹⁰) monocyclic groups and particularly saturated monocyclic groups, e.g. cycloalkyl groups. Examples of such cycloalkyl groups include cyclopropyl, cyclobutyl, cyclopentyl, cyclohexyl and cycloheptyl; more typically cyclopropyl, cyclobutyl, cyclopentyl and cyclohexyl, particularly cyclohexyl.

Further examples of non-aromatic cyclic groups include bridged ring systems such as bicycloalkanes and azabicycloalkanes although such bridged ring systems are generally less preferred. By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged ring systems include bicyclo[2.2.1]heptane, aza-bicyclo[2.2.1]heptane, bicyclo[2.2.2]octane, aza-bicyclo[2.2.2]octane, bicyclo[3.2.1]octane and aza-bicyclo[3.2.1]octane.

Where reference is made herein to carbocyclic and heterocyclic groups, the carbocyclic or heterocyclic ring can, unless the context indicates otherwise, be unsubstituted or substituted by one or more substituent groups R¹⁰ selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; or two adjacent groups R¹⁰, together with the carbon atoms or heteroatoms to which they are attached may form a 5-membered heteroaryl ring or a 5- or 6-membered non-aromatic heterocyclic ring, wherein the said heteroaryl and heterocyclic groups contain up to 3 heteroatom ring members selected from N, O and S;
R^{c} is selected from hydrogen and C₁₋₄ hydrocarbyl; and

X¹ is O, S or NR^{c} and X² is ═O, ═S or ═NR^{c}.

Where the substituent group R¹⁰ comprises or includes a carbocyclic or heterocyclic group, the said carbocyclic or heterocyclic group may be unsubstituted or may itself be substituted with one or more further substituent groups R¹⁰. In one sub-group of compounds of the formula (I), such further substituent groups R¹⁰ may include carbocyclic or heterocyclic groups, which are typically not themselves further substituted. In another sub-group of compounds of the formula (I), the said further substituents do not include carbocyclic or heterocyclic groups but are otherwise selected from the groups listed above in the definition of R¹⁰.

The substituents R¹⁰ may be selected such that they contain no more than 20 non-hydrogen atoms, for example, no more than 15 non-hydrogen atoms, e.g. no more than 12, or 10, or 9, or 8, or 7, or 6, or 5 non-hydrogen atoms.

Where the carbocyclic and heterocyclic groups have a pair of substituents on adjacent ring atoms, the two substituents may be linked so as to form a cyclic group. For example, an adjacent pair of substituents on adjacent carbon atoms of a ring may be linked via one or more heteroatoms and optionally substituted alkylene groups to form a fused oxa-, dioxa-, aza-, diaza- or oxa-aza-cycloalkyl group. Examples of such linked substituent groups include:

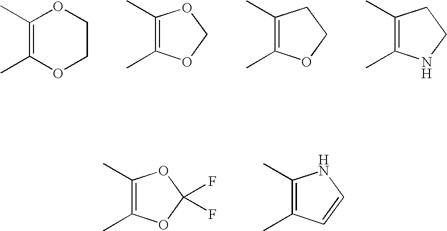

Examples of halogen substituents include fluorine, chlorine, bromine and iodine. Fluorine and chlorine are particularly preferred.

In the definition of the compounds of the formula (I) above and as used hereinafter, the term “hydrocarbyl” is a generic term encompassing aliphatic, alicyclic and aromatic groups having an all-carbon backbone and consisting of carbon and hydrogen atoms, except where otherwise stated. The hydrocarbyl groups may be saturated or unsaturated, the term “saturated” meaning that the hydrocarbyl contains no multiple bonds between adjacent carbon atoms, and the term “unsaturated” meaning that at least one pair of adjacent carbon atoms in the group is linked by a multiple bond and/or the hydrocarbyl group has aromatic character.

Examples of hydrocarbyl groups include saturated groups such as alkyl and cycloalkyl, and groups having varying degrees of unsaturation such as cycloalkenyl, aryl, alkenyl, alkynyl, cycloalkylalkyl, cycloalkenylalkyl, aralkyl, aralkenyl and aralkynyl groups. Such groups can be unsubstituted or, where stated, can be substituted by one or more substituents as defined herein. In certain cases, as defined herein, one or more of the carbon atoms making up the carbon backbone may be replaced by a specified atom or group of atoms. The examples and preferences expressed below apply to each of the hydrocarbyl substituent groups or hydrocarbyl-containing substituent groups referred to in the various definitions of substituents for compounds of the formula (I) and sub-groups thereof as defined herein unless the context indicates otherwise.

Generally by way of example, the hydrocarbyl groups can have up to eight carbon atoms, unless the context requires otherwise. Within the sub-set of hydrocarbyl groups having 1 to 8 carbon atoms, particular examples are C₁₋₆ hydrocarbyl groups, such as C₁₋₄ hydrocarbyl groups (e.g. C₁₋₃ hydrocarbyl groups or C₁₋₂ hydrocarbyl groups), specific examples being any individual value or combination of values selected from C₁, C₂, C₃, C₄, C₅, C₆, C₇ and C₈ hydrocarbyl groups.

The term “saturated hydrocarbyl”, whether used alone or together with a suffix such as “oxy” (e.g. as in “hydrocarbyloxy”), refers to a non-aromatic hydrocarbon group (e.g. alkyl and cycloalkyl) containing no multiple bonds such as C═C and C≡C.

The term “alkyl” covers both straight chain and branched chain alkyl groups. Examples of alkyl groups include methyl, ethyl, propyl, isopropyl, n-butyl, isobutyl, tert-butyl, n-pentyl, 2-pentyl, 3-pentyl, 2-methyl butyl, 3-methyl butyl, and n-hexyl and its isomers. Within the sub-set of alkyl groups having 1 to 8 carbon atoms, particular examples are C₁₋₆ alkyl groups, such as C₁₋₄ alkyl groups (e.g. C₁₋₃ alkyl groups or C₁₋₂ alkyl groups).

Examples of cycloalkyl groups are those derived from cyclopropane, cyclobutane, cyclopentane, cyclohexane and cycloheptane. Within the sub-set of cycloalkyl groups the cycloalkyl group will have from 3 to 8 carbon atoms, particular examples being C₃₋₆ cycloalkyl groups.

Examples of alkenyl groups include, but are not limited to, ethenyl (vinyl), 1-propenyl, 2-propenyl (allyl), isopropenyl, butenyl, buta-1,4-dienyl, pentenyl, and hexenyl. Within the sub-set of alkenyl groups the alkenyl group will have 2 to 8 carbon atoms, particular examples being C₂₋₆ alkenyl groups, such as C₂₋₄ alkenyl groups.

Examples of cycloalkenyl groups include, but are not limited to, cyclopropenyl, cyclobutenyl, cyclopentenyl, cyclopentadienyl and cyclohexenyl. Within the sub-set of cycloalkenyl groups the cycloalkenyl groups have from 3 to 8 carbon atoms, and particular examples are C₃₋₆ cycloalkenyl groups.

Examples of alkynyl groups include, but are not limited to, ethynyl and 2-propynyl (propargyl) groups. Within the sub-set of alkynyl groups having 2 to 8 carbon atoms, particular examples are C₂₋₆ alkynyl groups, such as C₂₋₄ alkynyl groups.

Examples of carbocyclic aryl groups (aryl hydrocarbyl groups) include unsubstituted phenyl and substituted phenyl (e.g. phenyl substituted by alkyl groups, such as toluene, xylene and mesitylene groups), and unsubstituted and substituted naphthyl, indane and indene groups.

Examples of cycloalkylalkyl, cycloalkenylalkyl, carbocyclic aralkyl, aralkenyl and aralkynyl groups include phenethyl, benzyl, styryl, phenylethynyl, cyclohexylmethyl, cyclopentylmethyl, cyclobutylmethyl, cyclopropylmethyl and cyclopentenylmethyl groups.

When present, and where stated, a hydrocarbyl group can be optionally substituted by one or more substituents selected from hydroxy, oxo, alkoxy, carboxy, halogen, cyano, nitro, amino, mono- or di-C₁₋₄ hydrocarbylamino, and monocyclic or bicyclic carbocyclic and heterocyclic groups having from 3 to 12 (typically 3 to 10 and more usually 5 to 10) ring members. Preferred substituents include halogen such as fluorine. Thus, for example, the substituted hydrocarbyl group can be a partially fluorinated or perfluorinated group such as difluoromethyl or trifluoromethyl. In one embodiment preferred substituents include monocyclic carbocyclic and heterocyclic groups having 3-7 ring members.

Where stated, one or more carbon atoms of a hydrocarbyl group may optionally be replaced by O, S, SO, SO₂, NR^{c}, X¹C(X²), C(X²)X¹ or X¹C(X²)X¹ (or a sub-group thereof) wherein X¹ and X² are as hereinbefore defined, provided that at least one carbon atom of the hydrocarbyl group remains. For example, 1, 2, 3 or 4 carbon atoms of the hydrocarbyl group may be replaced by one of the atoms or groups listed, and the replacing atoms or groups may be the same or different. In general, the number of linear or backbone carbon atoms replaced will correspond to the number of linear or backbone atoms in the group replacing them. Examples of groups in which one or more carbon atom of the hydrocarbyl group have been replaced by a replacement atom or group as defined above include ethers and thioethers (C replaced by O or S), amides, esters, thioamides and thioesters (C—C replaced by X¹C(X²) or C(X²)X¹), sulphones and sulphoxides (C replaced by SO or SO₂), amines (C replaced by NR^{c}). Further examples include ureas, carbonates and carbamates (C—C—C replaced by X¹C(X²)X¹).

Where an amino group has two hydrocarbyl substituents, they may, together with the nitrogen atom to which they are attached, and optionally with another heteroatom such as nitrogen, sulphur, or oxygen, link to form a ring structure of 4 to 7 ring members.

The term “aza-cycloalkyl” as used herein refers to a cycloalkyl group in which one of the carbon ring members has been replaced by a nitrogen atom. Thus examples of aza-cycloalkyl groups include piperidine and pyrrolidine. The term “oxa-cycloalkyl” as used herein refers to a cycloalkyl group in which one of the carbon ring members has been replaced by an oxygen atom. Thus examples of oxa-cycloalkyl groups include tetrahydrofuran and tetrahydropyran. In an analogous manner, the terms “diaza-cycloalkyl”, “dioxa-cycloalkyl” and “aza-oxa-cycloalkyl” refer respectively to cycloalkyl groups in which two carbon ring members have been replaced by two nitrogen atoms, or by two oxygen atoms, or by one nitrogen atom and one oxygen atom.

The definition “R^{a}-R^{b}” as used herein, either with regard to substituents present on a carbocyclic or heterocyclic moiety, or with regard to other substituents present at other locations on the compounds of the formula (I), includes inter alia compounds wherein R^{a} is selected from a bond, O, CO, OC(O), SC(O), NR^{c}C(O), OC(S), SC(S), NR^{c}C(S), OC(NR^{c}), SC(NR^{c}), NR^{c}C(NR^{c}), C(O)O, C(O)S, C(O)NR^{c}, C(S)O, C(S)S, C(S)NR^{c}, C(NR^{c})O, C(NR^{c})S, C(NR^{c})NR^{c}, OC(O)O, SC(O)O, NR^{c}C(O)O, OC(S)O, SC(S)O, NR^{c}C(S)O, OC(NR^{c})O, SC(NR^{c})O, NR^{c}C(NR^{c})O, OC(O)S, SC(O)S, NR^{c}C(O)S, OC(S)S, SC(S)S, NR^{c}C(S)S, OC(NR^{c})S, SC(NR^{c})S, NR^{c}C(NR^{c})S, OC(O)NR^{c}, SC(O)NR^{c}, NR^{c}C(O)NR^{c}, OC(S)NR^{c}, SC(S)NR^{c}, NR^{c}C(S)NR^{c}, OC(NR^{c})NR^{c}, SC(NR^{c})NR^{c}, NR^{c}C(NR^{c}NR^{c}, S, SO, SO₂, NR^{c}, SO₂NR^{c} and NR^{c}SO₂ wherein R^{c} is as hereinbefore defined.

The moiety R^{b} can be hydrogen or it can be a group selected from carbocyclic and heterocyclic groups having from 3 to 12 ring members (typically 3 to 10 and more usually from 5 to 10), and a C₁₋₈ hydrocarbyl group optionally substituted as hereinbefore defined. Examples of hydrocarbyl, carbocyclic and heterocyclic groups are as set out above.

When R^{a} is O and R^{b} is a C₁₋₈ hydrocarbyl group, R^{a} and R^{b} together form a hydrocarbyloxy group. Preferred hydrocarbyloxy groups include saturated hydrocarbyloxy such as alkoxy (e.g. C₁₋₆ alkoxy, more usually C₁₋₄ alkoxy such as ethoxy and methoxy, particularly methoxy), cycloalkoxy (e.g. C₃₋₆ cycloalkoxy such as cyclopropyloxy, cyclobutyloxy, cyclopentyloxy and cyclohexyloxy) and cycloalkyalkoxy (e.g. C₃₋₆ cycloalkyl-C₁₋₂ alkoxy such as cyclopropylmethoxy).

The hydrocarbyloxy groups can be substituted by various substituents as defined herein. For example, the alkoxy groups can be substituted by halogen (e.g. as in difluoromethoxy and trifluoromethoxy), hydroxy (e.g. as in hydroxyethoxy), C₁₋₂ alkoxy (e.g. as in methoxyethoxy), hydroxy-C₁₋₂ alkyl (as in hydroxyethoxyethoxy) or a cyclic group (e.g. a cycloalkyl group or non-aromatic heterocyclic group as hereinbefore defined). Examples of alkoxy groups bearing a non-aromatic heterocyclic group as a substituent are those in which the heterocyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkoxy group is a C₁₋₄ alkoxy group, more typically a C₁₋₃ alkoxy group such as methoxy, ethoxy or n-propoxy.

Alkoxy groups substituted by a monocyclic group such as pyrrolidine, piperidine, morpholine and piperazine and N-substituted derivatives thereof such as N-benzyl, N—C₁₋₄ acyl and N—C₁₋₄ alkoxycarbonyl. Particular examples include pyrrolidinoethoxy, piperidinoethoxy and piperazinoethoxy.

When R^{a} is a bond and R^{b} is a C₁₋₈ hydrocarbyl group, examples of hydrocarbyl groups R^{a}—R^{b} are as hereinbefore defined. The hydrocarbyl groups may be saturated groups such as cycloalkyl and alkyl and particular examples of such groups include methyl, ethyl and cyclopropyl. The hydrocarbyl (e.g. alkyl) groups can be substituted by various groups and atoms as defined herein. Examples of substituted alkyl groups include alkyl groups substituted by one or more halogen atoms such as fluorine and chlorine (particular examples including bromoethyl, chloroethyl, difluoromethyl, 2,2,2-trifluoroethyl and perfluoroalkyl groups such as trifluoromethyl), or hydroxy (e.g. hydroxymethyl and hydroxyethyl), C₁₋₈ acyloxy (e.g. acetoxymethyl and benzyloxymethyl), amino and mono- and dialkylamino (e.g. aminoethyl, methylaminoethyl, dimethylaminomethyl, dimethylaminoethyl and tert-butylaminomethyl), alkoxy (e.g. C₁₋₂ alkoxy such as methoxy—as in methoxyethyl), and cyclic groups such as cycloalkyl groups, aryl groups, heteroaryl groups and non-aromatic heterocyclic groups as hereinbefore defined).

Particular examples of alkyl groups substituted by a cyclic group are those wherein the cyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkyl group is a C₁₋₄ alkyl group, more typically a C₁₋₃ alkyl group such as methyl, ethyl or n-propyl. Specific examples of alkyl groups substituted by a cyclic group include pyrrolidinomethyl, pyrrolidinopropyl, morpholinomethyl, morpholinoethyl, morpholinopropyl, piperidinylmethyl, piperazinomethyl and N-substituted forms thereof as defined herein.

Particular examples of alkyl groups substituted by aryl groups and heteroaryl groups include benzyl and pyridylmethyl groups.

When R^{a} is SO₂NR^{c}, R^{b} can be, for example, hydrogen or an optionally substituted C₁₋₈ hydrocarbyl group, or a carbocyclic or heterocyclic group. Examples of R^{a}—R^{b} where R^{a} is SO₂NR^{c} include aminosulphonyl, C₁₋₄ alkylaminosulphonyl and di-C₁₋₄ alkylaminosulphonyl groups, and sulphonamides formed from a cyclic amino group such as piperidine, morpholine, pyrrolidine, or an optionally N-substituted piperazine such as N-methyl piperazine.

Examples of groups R^{a}—R^{b} where R^{a} is SO₂ include alkylsulphonyl, heteroarylsulphonyl and arylsulphonyl groups, particularly monocyclic aryl and heteroaryl sulphonyl groups. Particular examples include methylsulphonyl, phenylsulphonyl and toluenesulphonyl.

When R^{a} is NR^{c}, R^{b} can be, for example, hydrogen or an optionally substituted C₁₋₈ hydrocarbyl group, or a carbocyclic or heterocyclic group. Examples of R^{a}—R^{b} where R^{a} is NR^{c} include amino, C₁₋₄ alkylamino (e.g. methylamino, ethylamino, propylamino, isopropylamino, tert-butylamino), di-C₁₋₄ alkylamino (e.g. dimethylamino and diethylamino) and cycloalkylamino (e.g. cyclopropylamino, cyclopentylamino and cyclohexylamino).

### SPECIFIC EMBODIMENTS OF AND PREFERENCES FOR R¹ TO R⁷ AND X

In this section, the preferences, definitions, sub-definitions, sub-groups, embodiments and examples set out above in the General Preferences and Definitions section may also be applied to each of the specific embodiments and preferences described below for each of the moieties R^{a} to R¹⁰, R^{a}, R^{b}, R^{c}, X, X¹ and X² and any sub-definition, sub-group or embodiment thereof, unless the context indicates otherwise.

In the general formula (I), the groups R¹ and R² are the same or different and each is selected from hydrogen, C₁₋₃ saturated hydrocarbyl, halogen and cyano.

In one group of compounds of the invention, R¹ is selected from hydrogen, C₁₋₃ saturated hydrocarbyl and halogen.

In another embodiment, R² is selected from hydrogen, C₁₋₃ saturated hydrocarbyl and halogen.

In a further embodiment, R¹ and R² are the same or different and each is selected from hydrogen, saturated C₁₋₃ hydrocarbyl and halogen.

In general, where R¹ and/or R² is/are halogen, the halogen is preferably selected from chlorine and fluorine, chlorine being particularly preferred.

Where R¹ and/or R² is/are saturated C₁₋₃ hydrocarbyl, the hydrocarbyl group can be selected from methyl, ethyl, n-propyl, i-propyl and cyclopropyl, preferred groups being methyl and ethyl, with methyl being particularly preferred.

In general, it is preferred that the total number of carbon, halogen and nitrogen atoms making up the substituent groups R¹ and R² does not exceed 5. More particularly, the total number of carbon, halogen and nitrogen atoms making up the substituent groups R¹ and R² is in the range 0 to 4, for example 0, 1, 2 or 3.

Typically, no more than one of the substituent groups R¹ and R² is a halogen.

When a halogen (particularly chlorine) or cyano group is present as one of the groups R¹ and R², the other group is typically hydrogen or methyl.

In one group of compounds of the invention, R¹ is a halogen, preferably chlorine.

Particular combinations of groups R¹ and R² include: (a) R¹=chlorine & R²=methyl; (b) R¹=chlorine & R²=hydrogen; (c) R¹=hydrogen & R²=hydrogen; (d) R¹=methyl & R²=hydrogen; (e) R=cyano & R²=methyl; and (f) R¹=methyl & R²=cyano. A presently preferred combination is combination (a).

In the general formula (I), X is selected from C═O, C═S, C(═O)NE, C(═S)NH, C(═O)O, C(═O)S, C(═S)O and C(═S)S.

In one group of compounds of the invention, X is selected from C═O and C(═O)NH.

In another group of compounds of the invention, X is C(═O)NH.

In a further group of compounds of the invention, X is selected from C═S, C(═O)NH, C(═S)NH, C(═S)O and C(═S)S.

The group R³ is selected from aryl and heteroaryl groups having from 5 to 12 ring members. It is presently preferred that the group R³ is a monocyclic aryl group or a monocyclic heteroaryl group containing at least one nitrogen atom, for example up to three nitrogen atoms, preferably 0, 1 or 2 nitrogen atoms. Examples of such groups include groups selected from the monocyclic members of the list of specific heteroaryl groups set out above. Particular examples of groups R³ are phenyl, pyrazolyl, and thiadiazolyl (e.g. [1,3,4]-thiadiazolyl).

In one sub-group of compounds of the invention, the substituent R³ is a monocyclic aryl or heteroaryl group of 5 or 6 ring members wherein the aryl or heteroaryl group bears a substituent group which is a 4-7 membered carbocylic and heterocyclic group. The carbocyclic or heterocyclic substituent can be linked to the aryl or heteroaryl group via a carbon-nitrogen bond.

The carbon atom of the carbon-nitrogen bond can form part of the aryl or heteroaryl group, or the carbon atom of the carbon-nitrogen bond can form part of the substituent group.

When the carbon atom of the carbon-nitrogen bond forms part of the substituent group, the substituent group can be for example an optionally substituted phenyl ring attached to the heteroaryl group via a nitrogen atom in the heteroaryl group.

The optional substituents on the phenyl ring may be selected from the list set out above in relation to R¹⁰. A preferred substituent is fluoro, for example para-fluoro.

When the nitrogen atom of the carbon-nitrogen bond forms part of the substituent group, the substituent group can be, for example, a 4 to 7 membered (more typically 5 to 6 membered) heterocyclic group R⁸ containing at least one nitrogen atom.

Preferred heterocyclic groups in this context include morpholino, piperidino, piperazino, N-methyl piperazino and pyrrolidino, with morpholino being particularly preferred.

Where the group R³ is a phenyl group, it can be optionally substituted by one or more substituents R¹⁰ as hereinbefore defined. One sub-group of compounds is the group of compounds wherein the phenyl ring contains one or two meta substituents, for example wherein one meta position on the phenyl ring is unsubstituted or is substituted by a group selected from fluorine, chorine, methoxy, trifluoromethoxy, trifluoromethyl, ethyl, methyl and isopropyl; and the other meta position is substituted by a group selected from fluorine, chorine, methoxy, trifluoromethoxy, trifluoromethyl, ethyl, methyl, isopropyl, isobutyl, t-butyl, phenyl, substituted phenyl, and five and six membered monocyclic heterocyclic groups.

One particular combination of meta substituents is the combination of a halogen, preferably fluoro, and a group R⁸ as hereinbefore defined.

Where the group R³ is a heteroaryl group, it can be, for example, a pyrazole group optionally substituted by one or more substituents R¹⁰ as hereinbefore defined. The pyrazole group can have, for example, one or two such substituent groups R¹⁰. Where there are two substituent groups R¹⁰ present, it is preferred that they are located on non-adjacent ring members. It is further preferred that at least one of the substituents is located at a position meta or β with respect to the ring member linked to the group X.

One particularly preferred group of compounds is the group wherein the heteroaryl group R³ is a pyrazolyl ring substituted by an optionally substituted phenyl group (e.g. 4-fluorophenyl) and a C₁₋₄ hydrocarbyl group, e.g. a tert-butyl group or a tert-butyl isostere.

Another particularly preferred group of compounds is the group wherein the heteroaryl group R³ is a thiadiazole group (e.g. a [1,3,4]-thiadiazole group).

The moieties R⁴ and R⁵ are the same or different and are selected from hydrogen and methyl; or one of R⁴ and R⁵ is selected from hydroxymethyl and ethyl and the other is hydrogen.

In one embodiment, R⁴ is hydrogen.

In another embodiment, R⁵ is hydrogen.

In a further embodiment, R⁴ and R⁵ are both hydrogen.

The moieties R⁶ and R⁷ are the same or different and are selected from hydrogen and methyl.

In one embodiment, R⁶ is hydrogen.

In another embodiment, R⁷ is hydrogen.

In a further embodiment, R⁶ and R⁷ are both hydrogen.

One group of compounds for use according to the invention is defined by the general formula (II); 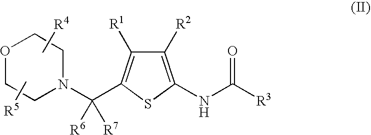
wherein R¹, R², R³, R⁴, R⁵, R⁶ and R⁷ are as hereinbefore defined.

Another group of compounds for use according to the invention is represented by the formula (III): 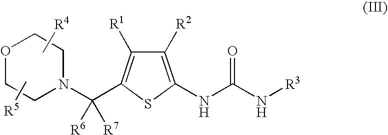
wherein R¹ to R⁷ are as hereinbefore defined.

Within the group of compounds of the formula (III) are compounds of the formula (IV): 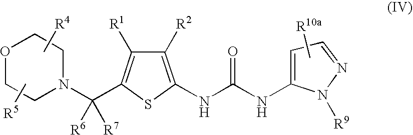
wherein R¹, R², R⁴, R⁵, R⁶ and R⁷ are as hereinbefore defined;

R⁹ is selected from carbocyclic and heterocyclic groups having from 3 to 7 ring members; a group R^{e}—R^{f} wherein R^{e} is a bond, CO, X¹C(X²), C(X²)X¹, X¹C(X²)X¹, SO, SO₂, SO₂NR^{c} or NR^{c}SO₂; and R^{f} is selected from (a) hydrogen, (b) carbocyclic and heterocyclic groups having from 3 to 7 ring members, and (c) a C₁₋₈ hydrocarbyl group optionally substituted by one or more substituents selected from hydroxy, oxo, halogen, cyano, nitro, amino, mono- or di-C₁₋₄ hydrocarbylamino, and carbocyclic and heterocyclic groups having from 3 to 7 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; where X¹, X² and R^{c} are as hereinbefore defined; and
R^{10a} is selected from hydrogen, halogen and C₁₋₆ hydrocarbyl optionally substituted by one or more substituents selected from hydroxy, oxo, halogen, cyano, nitro, and wherein one or more carbon atoms of the C₁₋₆ hydrocarbyl group may optionally be replaced by O, S, SO, SO₂, NR^{c}, X¹C(X²), C(X²)X¹ or X¹C(X²)X¹; where X¹, X² and R^{c} are as hereinbefore defined.

In the group of compounds defined by formula (IV), R⁹ is preferably a phenyl group, for example a fluorophenyl group (e.g. a 4-fluorophenyl group); and R^{10a} is preferably a hydrogen atom or a C₁₋₆ alkyl group, particular examples of which are methyl, ethyl, propyl, isopropyl, butyl, isobutyl and tertiary butyl; with tertiary butyl being particularly preferred.

A further group of compounds within the general formula (III) is the group of compounds of the formula (V): 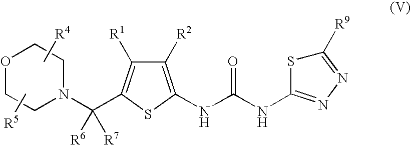
wherein R¹¹ is R⁶ or NHR⁵; and R¹, R², R⁵, R⁶ and R⁹ are as hereinbefore defined.

Another group of compounds of the formula (II) is defined by formula (VI): 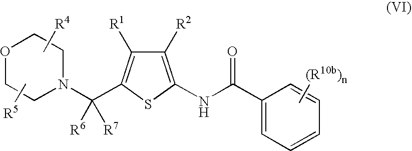
where n is 0-3, preferably 0-2, and more preferably 1 or 2, R^{10b} is a group R¹⁰ or R^{10a} and R¹, R², R⁴ to R⁷, R¹⁰ and R^{10a} are as hereinbefore defined.

In one embodiment of formula (VI), R^{10b} is a group R¹⁰ as hereinbefore defined, the reference to R¹⁰ including the preferences, sub-groups and examples thereof as set out herein.

In another embodiment, R^{10b} is a group R^{10a} as hereinbefore defined.

In one general embodiment, it is preferred that when X is C═O or C═S and R³ bears a substituent group R^{a}—R^{b} attached to an atom adjacent the atom in R³ to which X is attached, and R^{b} is a carbocyclic or heterocyclic group or C₁₋₈ hydrocarbyl substituted by a carbocyclic or heterocyclic group, then R^{a} is selected from a bond, O, CO, X¹C(X²)X¹, S, SO and SO₂.

In another general embodiment, it is preferred that when X is CO, R³ is other than a fused bicyclic aromatic or partially aromatic group bearing a substituent on a ring atom adjacent the ring atom to which X is attached.

For the avoidance of doubt, it is to be understood that each general and specific preference, embodiment and example of any one group selected from R¹, R², R³R⁴, R⁵, R⁶, R⁷, R⁸, R⁹ and R¹⁰ and sub-groups thereof may be combined with each general and specific preference, embodiment and example of any one or more other groups selected from R¹, R², R³ R⁴, R⁵, R⁶, R⁷, R⁸, R⁹ and R¹⁰ and sub-groups thereof and that all such combinations are embraced by this application.

The various functional groups and substituents making up the compounds of the formula (I) are typically chosen such that the molecular weight of the compound of the formula (I) does not exceed 1000. More usually, the molecular weight of the compound will be less than 750, for example less than 700, or less than 650, or less than 600, or less than 550. More preferably, the molecular weight is less than 525 and, for example, is 500 or less.

Specific examples of novel compounds within the scope of the present invention include:
N-(4-chloro-3-methyl-5-(morpholin-yl methyl-thiophen-2-yl)-3-fluoro-morpholin-4-yl-benzamide;
1-[5-tert-butyl-2(4-fluoro-phenyl)-2H-pyrazol-3-yl]-3-(4-chloro-3-methyl-5-morpholin-4-ylmethyl-thiophen-2-yl)urea;
1-[5-tert-butyl-2-(2,4-difluoro-phenyl)-2H-pyrazol-3-yl]-3-(4-chloro-3-methyl-5-morpholin-4-ylmethyl-thiophen-2-yl)-urea; and
1-(4-chloro-3-methyl-5-morpholin-4-ylmethyl-thiophen-2-yl)-3-[5-(tetrahydro-furan-2-yl)-[1,3,4]thiadiazol-2-yl]-urea.

Particular compounds of the invention are as illustrated in the examples below.

Salts, Solvates, Tautomers, Isomers, N-Oxides, Esters, Prodrugs and Isotopes

Unless otherwise specified, a reference to a particular compound also includes ionic, salt, solvate, and protected forms thereof, for example, as discussed below.

Many compounds of the formula (I) can exist in the form of salts, for example acid addition salts or, in certain cases salts of organic and inorganic bases such as carboxylate, sulphonate and phosphate salts. All such salts are within the scope of this invention, and references to compounds of the formula (I) include the salt forms of the compounds. As in the preceding sections of this application, all references to formula (I) should be taken to refer also to formulae (II), (III), (IV), (V), (VI) and sub-groups thereof unless the context indicates otherwise.

Salt forms may be selected and prepared according to methods described in Pharmaceutical Salts Properties, Selection, and Use, P. Heinrich Stahl (Editor), Camille G. Wermuth (Editor), ISBN: 3-90639-026-8, Hardcover, 388 pages, August 2002.

Acid addition salts may be formed with a wide variety of acids, both inorganic and organic. Examples of acid addition salts include salts formed with an acid selected from the group consisting of acetic, 2,2-dichloroacetic, adipic, alginic, ascorbic (e.g. L-ascorbic), L-aspartic, benzenesulphonic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrobromic, hydrochloric, hydriodic, isethionic, lacetic (e.g. (+)-L-lacetic and (±)-DL-lacetic), lactobionic, maleic, malic, (−)-L-malic, malonic, (±)-DL-mandelic, methanesulphonic, naphthalenesulphonic (e.g. naphthalene-2-sulphonic), naphthalene-1,5-disulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, talmic, (+)-L-tartaric, thiocyanic, toluenesulphonic (e.g. p-toluenesulphonic), uidecylenic and valeric acids, as well as acylated amino acids and cation exchange resins.

One particular group of acid addition salts consists of salts formed with hydrochloric, hydriodic, phosphoric, nitric, sulphuric, citric, lacetic, succinic, maleic, malic, isethionic, firmaric, benzenesulphonic, toluenesulphonic, methanesulphonic, ethanesulphonic, naphthalenesulphonic, valeric, acetic, propanoic, butanoic, malonic, glucuronic and lactobionic acids.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the formula (I) contain an amine function, these may form quaternary ammonium salts, for example by reaction with an alkylating agent according to methods well known to the skilled person. Such quaternary ammonium compounds are within the scope of formula (I).

The salt forms of the compounds of the invention are typically pharmaceutically acceptable salts, and examples of pharmaceutically acceptable salts are discussed in Berge et al., 1977, “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention.

Compounds of the formula (I) containing an amine function may also form N-oxides. A reference herein to a compound of the formula (I) that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with ni-chloroperoxybenzoic acid (MCPBA), for example, in an inert solvent such as dichloromethane.

Compounds of the formula (I) may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I).

Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

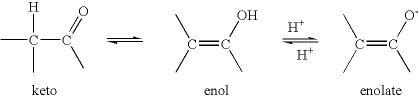

Where compounds of the formula (I) contain one or more chiral centres, and can exist in the form of two or more optical isomers, references to compounds of the formula (I) include all optical isomeric forms thereof (e.g. enantiomers, epimers and diastereoisomers), either as individual optical isomers, or mixtures (e.g. racemic mixtures) or two or more optical isomers, unless the context requires otherwise.

The optical isomers may be characterised and identified by their optical activity (i.e. as + and − isomers, or d and l isomers) or they may be characterised in terms of their absolute stereochemistry using the “R and S” nomenclature developed by Cahn, Ingold and Prelog, see Advanced Organic Chemistry by Jerry March, 4^{th} Edition, John Wiley & Sons, New York, 1992, pages 109-114, and see also Cahn, Ingold & Prelog, Angew. Chem. Int. Ed. Engl., 1966, 5, 385-415.

Optical isomers can be separated by a number of techniques including chiral chromatography (chromatography on a chiral support) and such techniques are well known to the person skilled in the art.

Where compounds of the formula (I) exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers. Accordingly, the invention provides compositions containing a compound of the formula (I) having one or more chiral centres, wherein at least 55% (e.g. at least 60%, 65%, 70%, 75%, 80%, 85%, 90% or 95%) of the compound of the formula (I) is present as a single optical isomer (e.g. enantiomer or diastereoisomer). In one general embodiment, 99% or more (e.g. substantially all) of the total amount of the compound of the formula (I) may be present as a single optical isomer (e.g. enantiomer or diastereoisomer).

The compounds of the invention include compounds with one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O.

The isotopes may be radioactive or non-radioactive. In one embodiment of the invention, the compounds contain no radioactive isotopes. Such compounds are preferred for therapeutic use. In another embodiment, however, the compound may contain one or more radioisotopes. Compounds containing such radioisotopes may be useful in a diagnostic context.

Esters such as carboxylic acid esters and acyloxy esters of the compounds of formula (I) bearing a carboxylic acid group or a hydroxyl group are also embraced by Formula (I). Examples of esters are compounds containing the group —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, —C(═O)OCH₃, —C(═O)OCH₂CH₃, —C(═O)OC(CH₃)₃, and —C(═O)OPh. Examples of acyloxy (reverse ester) groups are represented by —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Also encompassed by formula (I) are any polymorphic forms of the compounds, solvates (e.g. hydrates), complexes (e.g. inclusion complexes or clathrates with compounds such as cyclodextrins, or complexes with metals) of the compounds, and pro-drugs of the compounds. By “prodrugs” is meant for example any compound that is converted in vivo into a biologically active compound of the formula (I).

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR
wherein R is:
C₁₋₇alkyl
(e.g., -Me, -Et, -ipr, -iPr, -nBu, -sBu, -iBu, -tBu);
C₁₋₇-aminoalkyl
(e.g., aminoethyl; 2-(N,N-diethylamino)ethyl; 2-(4-morpholino)ethyl); and
acyloxy-C₁₋₇alkyl
(e.g., acyloxymethyl;
acyloxyethyl;
pivaloyloxymethyl;
acetoxymethyl;
1-acetoxyethyl;
1-(1-methoxy-1-methyl)ethyl-carbonxyloxyethyl;
1-(benzoyloxy)ethyl; isopropoxy-carbonyloxymethyl;
1-isopropoxy-carbonyloxyethyl; cyclohexyl-carbonyloxymethyl;
1-cyclohexyl-carbonyloxyethyl;
cyclohexyloxy-carbonyloxymethyl;
1-cyclohexyloxy-carbonyloxyethyl;
(4-tetrahydropyranyloxy)carbonyloxymethyl;
1-(4-tetrahydropyranyloxy)carbonyloxyethyl;
(4-tetrahydropyranyl)carbonyloxymethyl; and
1-(4-tetrahydropyranyl)carbonyloxyethyl).

Also, some prodrugs are activated enzymatically to yield the active compound, or a compound which, upon further chemical reaction, yields the active compound (for example, as in Antibody-directed Enzyme Prodrug Therapy (ADEPT), Gene-directed Enzyme Prodrug Therapy (GDEPT), Polymer-directed Enzyme Prodrug Therapy (PDEPT), Ligand-directed Enzyme Prodrug Therapy (LIDEPT), etc.). For example, the prodrug may be a sugar derivative or other glycoside conjugate, or may be an amino acid ester derivative.

Methods for the Preparation of Compounds of the Formula (I)

The compounds of the formula (I) can be made by the methods described below and in the examples and by methods well known to the skilled person. In this section, as in the other sections of this application, references to formula (I) include formulae (II), (III), (IV), (V) and (VI) and sub-groups, embodiments and examples thereof unless the context indicates otherwise. The moieties R¹ to R⁷ and X have the meanings set out in the preceding sections of this application.

Compounds of the formula (I) wherein R⁶ and R⁷ are hydrogen can be prepared by the S-alkylation (e.g. methylation) of a compound of the formula (X): 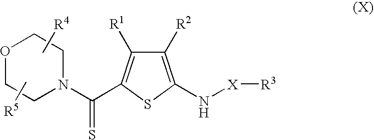
using an alkylating agent such as methyl iodide to give a thioimidate intermediate (not shown) which can then be reduced to the compound of formula (I) by means of a reducing agent such as a borohydride, preferably an alkali metal borohydride, e.g. sodium borohydride. The reduction of the thioimidate is typically carried out at ambient temperatures in an alcohol solvent such as methanol.

Alternatively, by treating the thioimidate intermediate with (i) methyl lithium or methylmagnesium bromide, followed by sodium borohydride, or (ii) two equivalents of methyl lithium or methylmagnesium bromide, using conditions analogous to those described in Arnat et al., J. Org. Chem., (2003) 1919-1928, Vol. 68, No. 5, compounds of the formula (I) in which R⁶ and/or R⁷ are methyl groups can be prepared.

The thioamide compound (X) can be prepared by the selective thionation of the morpholine-amide group in a compound of the formula (XI): 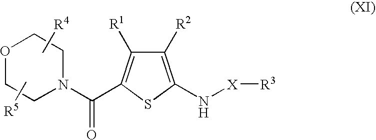
using a thionating agent such as phosphorus pentasulphide (P₂S₅) or a derivative thereof such as Lawesson's reagent under standard thionation conditions. The amide (XII) can be prepared by reacting a carboxylic acid of the formula (XII): 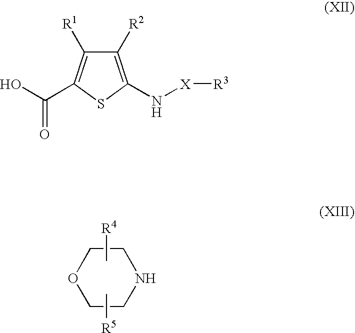
or an activated derivative thereof, with an optionally substituted morpholine compound of the formula (XIII).

The coupling reaction between the morpholine compound (XIII) and the carboxylic acid (XII) can be carried out by forming an activated derivative of the acid such as an acid chloride (e.g. by reaction with thionyl chloride), and then reacting the acid chloride with the amine, for example by the method described in Zh. Obs. Khim. 31, 201 (1961), and the method described in U.S. Pat. No. 3,705,175. Alternatively, acid chlorides can be formed by reacting the acid with oxalyl chloride the presence of dimethyl formamide, or by forming the carboxylate salt and reacting the salt with oxalyl chloride.

Alternatively, and more preferably, the coupling reaction between the carboxylic acid (XII) and the morpholine compound (XIII) can be carried out in the presence of an amide coupling reagent of the type commonly used to form peptide linkages.

Examples of such reagents include 1,3-dicyclohexylcarbodiimide (DCC) (Sheehan et al., J. Amer. Chem. Soc. 1955, 77, 1067), 1-ethyl-3-(3′-dimethylaminopropyl)-carbodiimide (EDAC) (Sheehan et al., J. Org. Chem., 1961, 26, 2525), uronium-based coupling agents such as O-(7-azabenzotriazol-1-yl)-N,N,N′,N′-tetramethyluronium hexafluorophosphate (HATU) and phosphonium-based coupling agents such as 1-benzo-triazolyloxytris-(pyrrolidino)phosphonium hexafluorophosphate (PyBOP) (Castro et al., Tetrahedron Letters, 1990, 31, 205). Carbodiimide-based coupling agents are advantageously used in combination with 1-hydroxy-7-azabenzotriazole (HOAt) (L. A. Carpino, J. Amer. Chem. Soc., 1993, 115, 4397) or 1-hydroxybenzotriazole (HOBt) (Konig et al., Chem. Ber., 103, 708, 2024-2034). Preferred coupling reagents include EDC and DCC in combination with HOAt or HOBt.

The coupling reaction is typically carried out in a non-aqueous, non-protic solvent such as dimethylsulfoxide, dichloromethane, dimethylformamide or N-methylpyrrolidine. The reaction can be carried out at room temperature or, where the reactants are less reactive (for example in the case of electron-poor anilines bearing electron withdrawing groups such as sulphonamide groups) at an appropriately elevated temperature. The reaction may be carried out in the presence of a non-interfering base, for example a tertiary amine such as triethylamine or N,N-diisopropylethylamine.

Compounds of the formula (XII) can be prepared by hydrolysis of a compound of the formula (XIV): 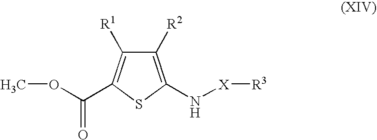
wherein R¹ to R³ are as hereinbefore defined. The hydrolysis reaction can be effected using standard methods, for example by treatment with an alkali metal hydroxide such as lithium hydroxide. The reaction is typically carried out in an aqueous solvent, optionally in the presence of a miscible co-solvent such as methanol or ethanol with heating to a non-extreme temperature between room temperature and 100° C., preferably a temperature below 80° C.

Compounds of the formula (XIV) in which X is CO can be prepared from compounds of the formula (XV): 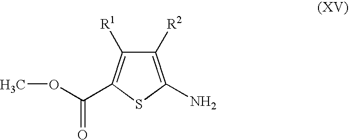
by reaction with a compound of the formula R³COOH or an activated derivative thereof such as an acid chloride in accordance with standard methods. Thus, for example, an acid chloride can be generated using oxalyl chloride and dimethylformamide in a non-protic solvent such as dichloromethane. Alternatively, coupling of the amine and carboxylic acid can be effected using one or more of the peptide coupling reagents described above.

Compounds of the formula (XIV) in which X is CONH, C(O)O and C(O)S can be prepared by reaction of a compound of the formula (VII) with a compound of the formula R³NH₂, R³OH, or R³SH and phosgene. The reaction is typically carried out in a non protic solvent such as dichloromethane or toluene, for example at a moderate temperature such as room temperature.

Compounds of the formula (XIV) in which X is C(═S)NH can be prepared by reacting a compound of the formula (XV) with an isothiocyanate R³NCS according to standard methods. Compounds of the formula (XIV) in which X is C(═S), C(═S)NH, C(═S)O and C(═S)S can be prepared from compounds of the formula (XVI): 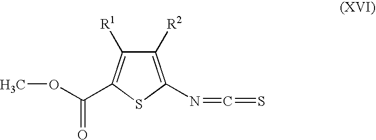
by reaction with a compound of the formula R³NH₂, R³⁰ or R³SO in accordance with standard methods. Examples of such methods can be found in Synthesis, Vol. 1, pp 108-118 (2001), Heterocyclic Chemistry, Vol. 17(8), pp 1789-92 (1980) and Zh. Org. Khim. Vol. 12(7), pp 1532-1535 (1976).

Compounds of the formula (XVI) can be prepared from the corresponding amine (XV) by reaction with thiophosgene, for example as described in Kryczka et al., Organiki, pp 65-72, 2001 and Grayson, Organic Process Research & Development, Vol. 1(3), pp 240-246 (1997).

Compounds of the formula (XV) are commercially available or can be prepared by nitration and reduction of a compound of the formula (XVII):

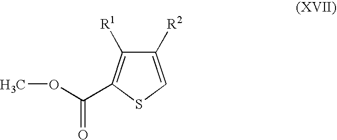

Nitration of the compound of the formula (XVII) can be achieved using standard conditions well known to the skilled chemist. For example, the compound of the formula (XVII) can be reacted with acetic acid and nitric acid in acetic anhydride, in the presence of a co-solvent, e.g. a halogenated hydrocarbon such as dichloromethane. Where required, the reaction mixture may be heated, for example to a temperature of up to about 100° C., more preferably up to about 80° C.

The resulting nitro-intermediate is reduced to give the amine using a suitable reducing agent. Thus, for example, reduction can be effected using a mixture of powdered iron and iron sulphate in an aqueous solvent optionally containing a water-miscible co-solvent such as dioxane.

Compounds of the formula (I) in which X is C(═O)NH can be prepared by reacting a compound of the formula (X): 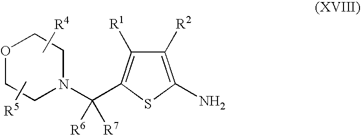
with phosgene and subsequently with a compound of the formula R³NH₂. The reaction is typically carried out in a dry aprotic solvent such as dichloromethane at a non-extreme temperature, for example at room temperature.

Compounds of the formula (XVIII) can be prepared by nitration of a compound of the formula (XIX) and subsequent reduction of the nitro group to an amino group.

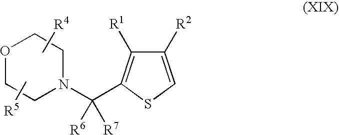

Nitration can be carried out using nitration conditions known to be suitable for nitrating thiophenes. For example, nitration may be effected using a nitronium salt such as nitronium tetrafluoroborate in a polar aprotic solvent such as acetonitrile. The reaction is typically carried out ambient temperatures or lower.

The compounds of the formula (XIX) can be prepared by reacting a carboxylic acid of the formula (XX) with a morpholine compound of the formula (XIII) using the methods of amide formation described above. 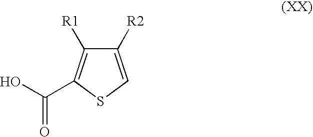
Novel Chemical Intermediates

Some of the intermediate compounds (in particular the thioamide compounds of the formula (X) above) used in the synthesis of the compounds of the formula (I) are novel and, as such, represent a further aspect of the invention.

Pharmaceutical Formulations

While it is possible for the active compound to be administered alone, it is preferable to present it as a pharmaceutical composition (e.g. formulation) comprising at least one active compound of the invention together with one or more pharmaceutically acceptable carriers, adjuvants, excipients, diluents, fillers, buffers, stabilisers, preservatives, lubricants, or other materials well known to those skilled in the art and optionally other therapeutic or prophylacetic agents.

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilizers, or other materials, as described herein.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Accordingly, in a further aspect, the invention provides compounds of the formula (I) and sub-groups thereof as defined herein in the form of pharmaceutical compositions.

The pharmaceutical compositions can be in any form suitable for oral, parenteral, topical, intranasal, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery.

In one preferred embodiment of the invention, the pharmaceutical composition is in a form suitable for i.v. administration, for example by injection or infusion.

In another preferred embodiment, the pharmaceutical composition is in a form suitable for sub-cutaneous (s.c.) administration.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Pharmaceutical compositions containing compounds of the formula (I) can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, e.g. lactose, sucrose, sorbitol or mannitol; and/or a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (e.g. tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. As a further alternative, the active compound can be formulated in a delivery system that provides osmotic control of the release of the compound. Osmotic release and other delayed release or sustained release formulations may be prepared in accordance with methods well known to those skilled in the art.

Compositions for topical use include ointments, creams, sprays, patches, gels, liquid drops and inserts (for example intraocular inserts). Such compositions can be formulated in accordance with known methods.

Compositions for parenteral administration are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the inventions will generally be presented in unit dosage form and, as such, will typically contain sufficient compound to provide a desired level of biological activity. For example, a formulation intended for oral administration may contain from 0.1 milligrams to 2 grams of active ingredient, more usually from 10 milligrams to 1 gram, for example, 50 milligrams to 500 milligrams.

The active compound will be administered to a patient in need thereof (for example a human or animal patient) in an amount sufficient to achieve the desired therapeutic effect.

Therapeutic Uses

Compounds of the formula (I) as defined above have activity in modulating or inhibiting p38 MAP kinase activity. As such, it is anticipated that the compounds possessing such activity will be useful therapeutic agents in the prophylaxis or treatment of diseases where the disease or condition is one in which the activity of p38 MAP kinase initiates or facilitates development of the disease Examples of conditions ameliorated by the inhibition of p38 MAP kinase are discussed above, and include, but are not limited to the said conditions. More particularly, the conditions can be selected from:

(i) inflammatory and arthritic diseases and conditions such as Reiter's syndrome, acute, synovitis, rheumatoid arthritis, osteoarthritis, rheumatoid spondylitis, gouty arthritis, traumatic arthritis, rubella arthritis, psoriatic arthritis, graft vs. host reaction and allograft rejections;

(ii) chronic inflammatory lung diseases such as emphysema, chronic pulmonary inflammatory disease, chronic obstructive pulmonary disease (COPD), adult respiratory distress syndrome and acute respiratory distress syndrome (ARDS);

(iii) lung diseases and conditions such as tuberculosis, silicosis, pulmonary sarcoidosis, pulmonary fibrosis and bacterial pneumonia;

(iv) inflammatory diseases and conditions of the enteric tract such as inflammatory bowel disease, Crohn's disease and ulcerative colitis;

(v) toxic shock syndrome and related diseases and conditions such as sepsis, septic shock, endotoxic shock, gram negative sepsis and the inflammatory reaction induced by endotoxin;

(vi) Alzheimer's disease;

(vii) reperfusion injury; and

(vii) diseases and conditions selected from atherosclerosis; muscle degeneration; gout; cerebral malaria; bone resorption diseases; fever and myalgias due to infection, such as influenza; cachexia, in particular cachexia secondary to infection or malignancy, cachexia secondary to acquired immune deficiency syndrome (AIDS); AIDS; ARC (AIDS related complex); keloid formation; scar tissue formation; pyresis and asthma.

Of particular interest are compounds for use in the treatment or prophylaxis of inflammatory diseases and conditions, rheumatoid arthritis and osteoarthritis.

Also of particular interest are compounds for use in the treatment or prophylaxis of chronic obstructive pulmonary disease (COPD).

Prevention or Treatment of Proliferative Disorders

The compounds of the formula (I) and sub-groups thereof are also expected to be useful in providing a means of preventing the growth or inducing apoptosis of neoplasias. It is therefore anticipated that the compounds will prove useful in treating or preventing proliferative disorders such as cancers.

Thus, it is envisaged that the compounds of the invention will be useful in the treatment or prophylaxis of any one more cancers selected from:

adenomas;

carcinomas;

leukaemias;

lymphomas;

melanomas;

sarcomas; and

teratomas.

Particular examples of cancers which may be inhibited include, but are not limited to, a carcinoma, for example a carcinoma of the bladder, breast, colon (e.g. colorectal carcinomas such as colon adenocarcinoma and colon adenoma), kidney, epidermal, liver, lung, for example adenocarcinoma, small cell lung cancer and non-small cell lung carcinomas, oesophagus, gall bladder, ovary, pancreas e.g. exocrine pancreatic carcinoma, stomach, cervix, thyroid, prostate, or skin, for example squamous cell carcinoma; a hematopoietic tumour of lymphoid lineage, for example leukaemia, acute lymphocytic leukaemia, B-cell lymphoma, T-cell lymphoma, Hodgkin's lymphoma, non-Hodgkin's lymphoma, hairy cell lymphoma, or Burkett's lymphoma; a hematopoietic tumour of myeloid lineage, for example acute and chronic myelogenous leukaemias, myelodysplastic syndrome, or promyelocytic leukaemia; thyroid follicular cancer; a tumour of mesenchymal origin, for example fibrosarcoma or habdomyosarcoma; a tumour of the central or peripheral nervous system, for example astrocytoma, neuroblastoma, glioma or schwannoma; melanoma; seminoma; teratocarcinoma; osteosarcoma; xenoderoma pigmentosum; keratoctanthoma; thyroid follicular cancer; or Kaposi's sarcoma.

One subset of cancers includes any one or more cancers selected from:

breast cancer;

ovarian cancer;

colon cancer;

prostate cancer;

oesophageal cancer;

squamous cancer;

and non-small cell lung carcinomas.

Another subset of cancers which are envisaged as being particularly susceptible to compounds of the invention that have raf kinase inhibitory activity includes breast cancer, ovarian cancer, colon cancer, melanoma, prostate cancer, oesophageal cancer, squamous cancer and non-small cell lung carcinomas.

A further subset of cancers that may be susceptible to raf kinase inhibitor compounds of the invention includes leukemia, chronic myelogenous leukemia and myelodysplastic syndrome.

For those compounds of the invention that are inhibitors of raf kinase, tumours with activating mutants of ras or overexpression of ras may be particularly sensitive to such raf inhibitors. Patients with activating mutants of any of the 3 isoforms of raf may also find treatment with raf inhibitors particularly beneficial. Tumours which have other abnormalities leading to an upregulated raf-MEK-ERK pathway signal may also be particularly sensitive to inhibitors of raf kinase. Examples of such abnormalities include but are not limited to constitutive activation of a growth factor receptor, overexpression of one or more growth factor receptors, overexpression of one or more growth factors, or other mutations or abnormalities leading to upregulation of the pathway.

Compounds of the invention are also provided for the treatment or prevention of inappropriate, excessive or undesirable angiogenesis. Diseases or conditions associated with inappropriate, excessive or undesirable angiogenesis are discussed in the “Background” section above. Of particular interest are conditions (e.g. cancer) characterised by the up-regulation of a receptor tyrosine kinase, such as FGFR-1, FGFR-2, FGFR-3, Tie2, VEGFR-2 and/or EphB2.

Compounds of the formula (I) that are inhibitors of receptor tyrosine kinase activity are expected to be useful in providing a means of preventing the growth or inducing apoptosis of neoplasias, particularly by inhibiting angiogenesis. It is therefore anticipated that the compounds will prove useful in treating or preventing proliferative disorders such as cancers. In particular tumours with activating mutants of receptor tyrosine kinases or upregulation of receptor tyrosine kinases may be particularly sensitive to the inhibitors. Patients with activating mutants of any of the isoforms of the specific RTKs discussed herein may also find treatment with RTK inhibitors particularly beneficial.

Methods of Diagnosis and Screening

Prior to administration of a compound of the formula (I), a patient may be screened to determine whether a disease or condition from which the patient is or may be suffering is one which would be susceptible to treatment with a compound having activity against raf kinases. For example, a biological sample taken from a patient may be analysed to determine whether a condition or disease, such as cancer, that the patient is or may be suffering from is one which is characterised by elevated expression, activation of a raf kinase (e.g. B-raf or C-raf) or the result of an activating mutation. Thus, the patient may be subjected to a diagnostic test to detect a marker characteristic of over-expression or activation of raf kinase or a mutation thereof.

The term “marker” include genetic markers including, for example, the measurement of DNA composition to identify mutations of raf, ras, MEK, ERK or a growth factor such as ERB2 or EGFR. The term “marker” also includes markers which are characteristic of up regulation of raf, ras, MEK, ERK, growth factors such as ERB2 or EGFR including enzyme activity, enzyme levels, enzyme state (e.g. phosphorylated or not) and mRNA levels of the aforementioned proteins.

Methods of identification and analysis of mutations are well known to a person skilled in the art, but typically include methods such as those described in Anticancer Research. 1999 19(4A) 2481-3, Clin Chem. 2002 48, 428 and Cancer Res. 2003 63(14) 3955-7 incorporated herein by reference.

Other tumours which have an up regulated raf-MEK-ERK pathway signal may also be particularly sensitive to inhibitors of raf kinases. A number of assays exist which can identify tumours which exhibit an up regulation in the raf-MEK-ERK pathway, including the commercially available MEK1/2 (MAPK Kinase) assay from Chemicon International. Up regulation can result from over expression or activation of growth factor receptors such as ERB2 and EGFR, or mutant ras or raf proteins.

Typical methods for screening for over expression, up regulation or mutants include, but are not limited to, standard methods such as reverse-transcriptase polymerase chain reaction (RT-PCR) or in-situ hybridisation.

In screening by RT-PCR, the level of mRNA for the aforementioned proteins in the tumour is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. Current Protocols in Molecular Biology, 2004, John Wiley & Sons Inc., or Innis, M. A. et-al., eds. PCR Protocols: a guide to methods and applications, 1990, Academic Press, San Diego. Reactions and manipulations involving nucleic acid techniques are also described in Sambrook et al., 2001, 3^{rd} Ed, Molecular Cloning: A Laboratory Manual, Cold Spring Harbor Laboratory Press. Alternatively a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference.

An example of an in-situ hybridisation technique would be fluorescence in-situ hybridisation (FISH) (see Angerer, 1987 Meth. Enzymol., 152: 649). Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al., eds. Current Protocols in Molecular Biology, 2004, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Alternatively, the protein products expressed from the mRNAs may be assayed by inimnunohistochemistry of tumour sections, solid phase immunoassay with microtiter plates, Western blotting, 2-dimensional SDS-polyacrylamide gel electrophoresis, ELISA, and other methods known in the art for detection of specific proteins. Detection methods would include the use of site specific antibodies, such as, phospho raf, phospho ERK or phospho MEK. Inaddition to tumour biopsies other samples which could be utilised include pleural fluid, peritoneal fluid, urine, stool biopsies, sputum, blood (isolation and enrichment of shed tumour cells).

In addition, mutant forms of raf, EGFR or ras can be identified by direct sequencing of, for example, tumour biopsies using PCR and methods to sequence PCR products directly as hereinbefore described. The skilled artisan will recognize that all such well-known techniques for detection of the over expression, activation or mutations of the aforementioned proteins could be applicable in the present case.

Finally, abnormal levels of proteins such as raf, ras and EGFR can be measured using standard enzyme assays, for example for raf those assays described herein.

Prior to administration of a receptor tyrosine kinase inhibitor of the formula (I), a patient may be screened to determine whether a disease or disease or condition from which the patient is or may be suffering is one which would be susceptible to treatment with a compound having activity against receptor tyrosine kinases. For example, a biological sample taken from a patient may be analysed to determine whether a condition or disease, such as cancer, that the patient is or may be suffering from is one which is characterised by elevated expression, activation of a receptor tyrosine kinase or the result of an activating mutation. Thus, the patient may be subjected to a diagnostic test to detect a marker characteristic of over-expression or activation of raf kinase or a mutation thereof.

The term “marker” include genetic markers including, for example, the measurement of DNA composition to identify mutations of RTKs, e.g. FGFR-1, FGFR-2, FGFR-3, VEGFR-2, Tie2 and EphB2. The term “marker” also includes markers which are characteristic of up regulation of RTKs, including enzyme activity, enzyme levels, enzyme state (e.g. phosphorylated or not) and mRNA levels of the aforementioned proteins.

Typical methods of screening for diseases or conditions caused by the up-regulation or mutants of FGFR, Tie, VEGFR and Eph kinases, include, but are not limited to, standard methods such as reverse-transcriptase polymerase chain reaction (RT-PCR) or in-situ hybridisation.

In screening by RT-PCR, the level of mRNA for the aforementioned proteins in tissue, such as tumour tissue is assessed by creating a cDNA copy of the mRNA followed by amplification of the cDNA by PCR. Methods of PCR amplification, the selection of primers, and conditions for amplification, are described above

Alternatively, the protein products expressed from the mRNAs may be assayed by immunohistochemistry of tumour sections, solid phase immunoassay with microtiter plates, Western blotting, 2-dimensional SDS-polyacrylamide gel electrophoresis, ELISA, and other methods known in the art for detection of specific proteins as described above. Detection methods would include the use of site specific antibodies, such as, phosphotyrosine. In addition to tumour biopsies other samples which could be utilised include pleural fluid, peritoneal fluid, urine, stool biopsies, sputum, blood (isolation and enrichment of shed tumour cells).

In addition, mutant forms of, for example, FGFR can be identified by direct sequencing of, for example, tumour biopsies using PCR and methods to sequence PCR products directly as hereinbefore described. Abnormal levels of proteins such as FGFR, Tie, VEGFR and Eph can be measured using standard enzyme assays, for example, those assays described herein.

Activation or overexpression could also be detected in a tissue sample, for example, a tumour tissue, by measuring the tyrosine kinase activity with an assay such as that from Chemicon International. The tyrosine kinase of interest would be immunoprecipitated from the sample lysate and its activity measured.

Alternative methods for the measurement of the over expression or activation of FGFR, Tie, VEGFR or Eph kinases, in particular VEGFR including the isoforms thereof, include the measurement of microvessel density. This can for example be measured using methods described by Orre and Rogers (Int J Cancer 1999 84(2) 101-8). Assay methods also include the use of markers, for example, in the case of VEGFR these include CD31, CD34 and CD105 (Mineo et al. J Clin Pathol. 2004 57(6) 591-7).

Methods of Treatment

Compounds of the formula (I) are generally administered to a subject in need of such administration, for example a human or animal patient, preferably a human.

The compounds will typically be administered in amounts that are therapeutically or prophylactically useful and which generally are non-toxic. However, in certain situations (for example in the case of life threatening diseases), the benefits of administering a compound of the formula (I) may outweigh the disadvantages of any toxic effects or side effects, in which case it may be considered desirable to administer compounds in amounts that are associated with a degree of toxicity.

The compounds may be administered over a prolonged term to maintain beneficial therapeutic effects or may be administered for a short period only. Alternatively they may be administered in a pulsatile manner.

A typical daily dose of the compound can be in the range from 100 picograms to 100 milligrams per kilogram of body weight, more typically 10 nanograms to 10 milligrams per kilogram of bodyweight although higher or lower doses may be administered where required. Ultimately, the quantity of compound administered will be commensurate with the nature of the disease or physiological condition being treated and will be at the discretion of the physician.

The compounds of the formula (I) can be administered as the sole therapeutic agent or they can be administered in combination therapy with one of more other compounds for treatment of a particular disease state.

For example, in the treatment of disease states or conditions mediated by p38 MAP kinase such as rheumatoid arthritis, osteoarthritis, chronic lung inflammatory diseases (e.g. COPD) and inflammatory bowel diseases, examples of other therapeutic agents that may be administered together (whether concurrently or at different time intervals) with the compounds of the formula (I) include methotrexate, prednisilone, sulfasalazine, leflunomide and NSAIDs, for example COX-2 inhibitors such as celecoxib, rofecoxib, valdecoxib and lumiracoxib, bronchodilators, e.g. beta agonists and anticholinergics such as salbutamol, salmeterol and ipatropium bromide; corticosteroids such as fluticasone proprionate; mucolytics such as guaifenesin; and antibiotics.

In the treatment of neoplastic diseases such as the cancers hereinbefore defined, examples of other therapeutic agents and methods that may be used or administered together (whether concurrently or at different time intervals) with the compounds of the formula (I) include but are not limited to:
Topoisomerase I inhibitors (for example camptothecin compounds such as topotecan (Hycamtin), irinotecan and CPT11 (Camptosar).
Antimetabolites (for example, anti-tumour nucleosides such as 5-fluorouracil, gemcitabine (Gemzar), raltitrexed (Tomudex), capecitabine (Xeloda), pemetrexed (Alimta), cytarabine or cytosine arabinoside or arabinosylcytosine [AraC] (Cytosar®), methotrexate (Matrex), fludarabine (Fludara) and tegafur.
Tubulin targeting agents (for example, vinca alkaloids, vinblastine and taxane compounds such as vincristine (Oncovin), vinorelbine (Navelbine), vinblastine (Velbe), paclitaxel (Taxol) and docetaxel (Taxotere).
DNA binder and topo II inhibitors (for example, podophyllo-toxin derivatives and anthracycline derivatives such as etoposide (Eposin, Etophos, Vepesid, VP-16), teniposide (Vumon), daunorubicin (Cerubidine, DaunoXome), epirubicin (Pharmorubicin), doxorubicin (Adriamycin; Doxil; Rubex), idarubicin (Zavedos), pegylated liposomal doxorubicin hydrochloride (Caeylx), liposome encapsulated doxorubicin citrate (Myocet), mitoxantrone (Novatrone, Onkotrone)
Alkylating Agents (for example, nitrogen mustard or nitrosourea alkylating agents and aziridines such as cyclophosphamide (Endoxana), melphalan (Alkeran), chlorambucil (Leukeran), busulphan (Myleran), carmustine (BiCNU), lomustine (CCNU), ifosfamide (Mitoxana), mitomycin (Mitomycin C Kyoma).
Alkylating Agents (for example, platinum compounds such as cisplatin, carboplatin (Paraplatin) and oxaliplatin (Eloxatin)
Monoclonal Antibodies (for example, the EGF family and its receptors and the VEGF family and its receptors, more particularly trastuzumab (Herceptin), cetuximab (Erbitux), rituximab (Mabthera), tositumomab (Bexxar), gemtuzumab ozogamicin (Mylotarg) and bevacizumab (Avastin).
Anti-Hormones (for example anti-androgens including anti-estrogen agents (e.g. aromatase inhibitors) such as tamoxifen (Nolvadex D, Soltamox, Tamofen), fulvestrant (Faslodex), raloxifene (Evista), toremifene (Fareston), droloxifene, letrazole (Femara), anastrazole (Arimidex), exemestane (Aromasin), vorozole (Rivizor), bicalutamide (Casodex, Cosudex), luprolide (Zoladex), megestrol acetate (Megace), aminoglutethimide (Cytadren) and bexarotene (Targretin).
Signal Transduction Inhibitors (such as gefitinib (Iressa), imatinib (Gleevec), erlotinib (Tarceva) and celecoxib (Celebrex).
Proteasome Inhibitors such as bortezimib (Velcade)
DNA methyl transferases such as temozolomide (Temodar)
Cytokines and retinoids such as interferon alpha (IntronA, Roferon-A), interleukin 2 (Aldesleukin, Proleukin) and all trans-retinoic acid [ATRA] or tretinoin (Vesanoid).
Radiotherapy.

Where the compounds of the invention are administered together with other therapeutic agents or therapeutic methods in a combination therapy, the two or more treatments may be given in individually varying dose schedules and via different routes.

Where the compound of the formula (I) is administered in combination therapy with one or more other therapeutic agents, the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s).

In the treatment of neoplastic diseases such as cancers, the compounds of the invention may also be administered in conjunction with non-chemotherapeutic treatments such as radiotherapy, photodynamic therapy, gene therapy; surgery and controlled diets.

For use in combination therapy with another chemotherapeutic agent, the compound of the formula (I) and one, two, three, four or more other therapeutic agents can be, for example, formulated together in a dosage form containing two, three, four or more therapeutic agents. In an alternative, the individual therapeutic agents may be formulated separately and presented together in the form of a kit, optionally with instructions for their use.

A person skilled in the art would know through their common general knowledge the dosing regimes and combination therapies to use.

### EXAMPLES

The invention will now be illustrated, but not limited, by reference to the specific embodiments described in the following examples.

In the examples, the compounds prepared were characterised by liquid chromatography and mass spectroscopy using two systems, the details of which are set out below. The two systems were equipped with identical chromatography columns and were set up to run under the same operating conditions. The operating conditions used are also described below.

1. Platform System

| | |
|---|---|
| System: | Waters 2790/Platform LC |
| Mass Spec Detector: | Micromass Platform LC |
| PDA Detector: | Waters 996 PDA |

Analytical Conditions:

| | |
|---|---|
| Eluent A: | H₂O (1% Formic Acid) |
| Eluent B: | CH₃CN (1% Formic Acid) |
| Gradient: | 5-95% eluent B |
| Flow: | 1.5 ml/min |
| Column: | Synergi 4 μm Max-RP C₁₂, 80 A, 50 × 4.6 mm (Phenomenex) |

MS Conditions:

| | | |
|---|---|---|
| Capillary voltage: | 3.5 | kV |
| Cone voltage: | 3 | V |

| | |
|---|---|
| Source Temperature: | 120 |

2. FractionLynx System

| | |
|---|---|
| System: | Waters FractionLynx (dual analytical/prep) |
| Mass Spec Detector: | Waters-Micromass ZQ |
| PDA Detector: | Waters 2996 PDA |

Analytical Conditions:

| | |
|---|---|
| Eluent A: | H₂O (1% Formic Acid) |
| Eluent B: | CH₃CN (1% Formic Acid) |
| Gradient: | 5-95% eluent B |
| Flow: | 1.5 ml/min |
| Column: | Synergi 4 μm Max-RP C₁₂, 80 A, 50 × 4.6 mm (Phenomenex) |

MS Conditions:

| | | |
|---|---|---|
| Capillary voltage: | 3.5 | kV |
| Cone voltage: | 30 | V |

| | |
|---|---|
| Source Temperature: | 120 |
| Desolvation Temperature: | 230 |

The starting materials for each of the Examples are commercially available unless otherwise specified.

### Example 1

### Preparation of N-(4-Chloro-3-methyl-5-(morpholin-yl methyl-thiophen-2-yl)-3-fluoro-morpholin-4-yl-benzamide

### 1A. Preparation of 3-fluoro-5-morpholin-4-yl-benzoic Acid

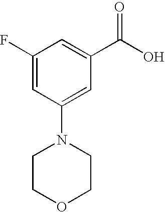

To a solution of 3,5-di-fluorobenzoic acid (commercially available) (10 g, 63.3 mmol) in ethanol (100 ml) was added concentrated sulphuric acid (5 ml) and the reaction was heated at 80° C. for 48 hours. The reaction mixture was evaporated and the residue was partitioned between ethyl acetate and 2N sodium hydroxide. The organic layer washed with saturated brine solution, dried (MgSO₄), filtered and evaporated to afford 3,5-di-fluorobenzoic acid ethyl ester as a pale yellow oil (8.79 g) which was used immediately in the next step without purification; δ_{H} (400 MHz, CDCl₃) 7.6 (m, 2H), 7.0 (m, 1H), 4.4 (q, 2H), 1.4 (t, 3H).

A mixture of 3,5-di-fluorobenzoic acid ethyl ester (8.79 g, 47.5 mmol) and morpholine (20 ml) in dimethylsulphoxide (250 ml) was heated at 100° C. with stirring for 3 days. The reaction was cooled and then partitioned between diethyl ether and water. The aqueous layer was extracted several times with diethyl ether and the organics were combined and dried over MgSO₄ before filtering the solution and evaporating the solvent under reduced pressure. The residue was subjected to purification by flash chromatography on silica gel. Eluting with 1:4 ethyl acetate: petroleum ether afforded 3-fluoro-5-morpholin-4-yl-benzoic acid ethyl ester as a yellow oil (4.8 g); δ_{H} (400 MHz, CDCl₃) 7.4 (s, 1H), 7.2 (d, 1H), 6.8 (d, 1H), 4.4 (q, 2H), 3.8 (t, 4H), 3.2 (t, 4H), 1.4(t, 3H).

A solution of 3-fluoro-5-morpholin-4-yl-benzoic acid ethyl ester (4.8 g, 18.9 mmol) in ethanol (20 ml) was treated with 2N sodium hydroxide (20 ml) and the reaction mixture was stirred at room temperature overnight. The reaction mixture was evaporated under reduced pressure and the residue was partitioned between ethyl acetate and water. The aqueous layer was acidified with 2N HCl and the solid precipitate was filtered, washed with diethyl ether and then dried to give the title compound as a white solid (3.1 g). LC MS-M+H 226

### 1B. Preparation of 3-chloro-4-methyl-5-aminothiophene-2-carboxylic Acid Methyl Ester

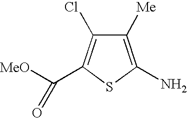

To a solution of 3-chloro-4-methyl-thiophene-2-carboxylic acid methyl ester (9 g, 47.37 mmol) in acetic anhydride (50 ml) and dichloromethane (70 ml) was added a mixture of acetic acid and concentrated nitric acid (5:1, 60 ml) at room temperature. The resulting solution was then heated to 80° C. for a period of 24 hours. Upon cooling, the solvent was removed under reduced pressure and the residue was dissolved in dichloromethane (250 ml). The organic solution washed with saturated sodium bicarbonate solution (50 ml) and brine (50 ml) before drying over MgSO₄. The resulting solution was filtered and the solvent was removed under reduced pressure to afford the crude product (12.9 g) which was used immediately in the next step without purification.

To a solution of the crude 3-chloro-4-methyl-5-nitrothiophene-2-carboxylic acid methyl ester (12.9 g, 54.9 mmol) in dioxane (250 ml) and water (50 ml) was added iron powder (27.6 g, 0.494 mol) followed by iron sulphate heptahydrate (33.6 g, 0.121 mol). The reaction mixture was then heated to reflux for 4 hours before cooling to room temperature. The solvent was then removed under reduced pressure and the residue was partitioned between ethyl acetate (150 ml) and 1N HCl (100 ml). The organic layer was separated and the aqueous layer was then basified with saturated sodium bicarbonate solution. The solution was extracted with ethyl acetate (2×250 ml), the organic layers were combined, dried (MgSO₄), filtered and the solvent was removed under reduced pressure. The residue was subjected to purification by flash column chromatography on silica gel, eluting with 15% ethyl acetate/petroleum ether to afford the title compound as an off white crystalline solid (1.77 g, 18% over two steps); LC MS M+H 206

### 1C. 3-Chloro-5-(3-fluoro-5-morpholin-4-yl-benzoylamino)-4-methyl-thiophene-2-carboxylic Acid Methyl Ester

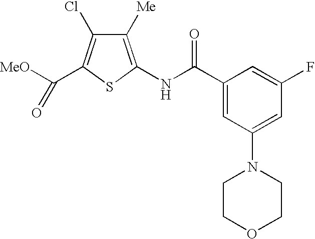

To a solution of 3-morpholino-5-fluorobenzoic acid (2.42 g, 10.75 mmol) in dichloromethane (100 ml) was added oxalyl chloride (1.11 ml, 12.90 mmol) followed by dimethylformamide (2 drops). The resulting solution was then stirred at room temperature, under an atmosphere of nitrogen, for a period of 4 hours. The solvent was then removed under reduced pressure and the residue was azeotroped to dryness by co-evaporation with toluene (2×50 ml). The resulting solid was then dissolved in dichloromethane (100 ml) and to the solution was added diisopropyl-ethylamine (5.62 ml, 32.19 mmol) followed by cautious addition of the aminothiophene product of Example 1B (2.2 g, 10.73 mmol). After stirring at room temperature under nitrogen for 17 hours, the reaction mixture was diluted with dichloromethane (150 ml) and partitioned with 1N HCl (50 ml). The organic layer was separated, washed successively with saturated sodium bicarbonate solution (50 ml) and brine (50 ml), dried (MgSO₄), filtered and concentrated. Purification by flash chromatography eluting with ethyl acetate/petroleum ether (1:4) gave the title compound as a white crystalline solid (1.60 g, 36%); LC MS M+H 413

### 1D. 3-Chloro-5-(3-Fluoro-5-morpholin-4-yl-benzoylamino)-4-methyl-thiophene-2-carboxylic Acid

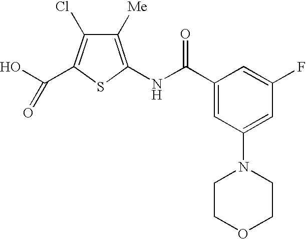

To a suspension of the ester product of Example 1C (0.903 g, 1.9 mmol) in methanol:water [2:1] (30 ml) was added lithium hydroxide (0.33 g, 7.6 mmol) and the reaction mixture was heated at 60° C. overnight. The solution was evaporated under reduced pressure and the residue was partitioned between ethyl acetate and water. The aqueous layer was acidified and extracted with ethyl acetate, dried (MgSO₄), filtered and evaporated under reduced pressure to give the crude title compound as an orange foam. (0.6 g); LC MS M+H 399

### 1E. Preparation of N-[4-Chloro-3-methyl-5-(morpholine-4-carbonyl)-thiophen-2-yl]-3-fluoro-5-morpholin-4-yl-benzamide

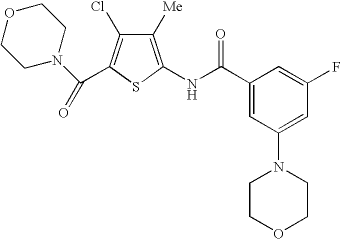

To a solution of the product of Example 1D, 3-chloro-5-(3-fluoro-5-morpholin-4-yl-benzoylamino)-4-methyl-thiophene-2-carboxylic acid, (100 mg, 0.25 mmol) in dimethylsulphoxide (2 ml) was added EDAC (72 mg, 0.37 mmol), HOAt (50 mg, 0.37 mmol) followed by morpholine (22 mg, 0.25 mmol). The reaction mixture was stirred at room temperature overnight, and the resultant solid was filtered and washed with methanol, affording the title product as an off-white solid (40 mg). LC MS M+H 469

### 1F. Preparation of N-[4-Chloro-3-methyl-5-(morpholine-4-carbothioyl)-thiophen-2-yl]-3-fluoro-5-morpholin-4-yl-benzamide

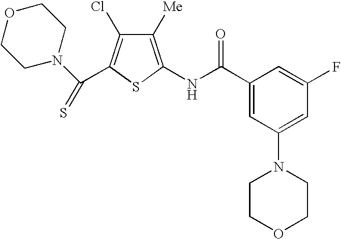

To a solution of the product of Example 1E, N-[4-Chloro-3-methyl-5-(morpholine-4-carbonyl)-thiophen-2-yl]-3-fluoro-5-morpholin-4-yl-benzamide, (225 mg, 0.48 mmol) in dry THF (40 ml) was added Lawesson's reagent (235 mg, 0.58 mmol). The reaction mixture was stirred at room temperature overnight and evaporated to dryness under reduced pressure. Purification by flash chromatography eluting with ethyl acetate/petroleum ether (1:5) gave the title compound as an orange solid (185 mg, 80%); LC MS M+H 484

### 1G. Preparation of N-(4-Chloro-3-methyl-5-(morpholin-yl methyl-thiophen-2-yl)-3-fluoro-morpholin-4-yl-benzamide

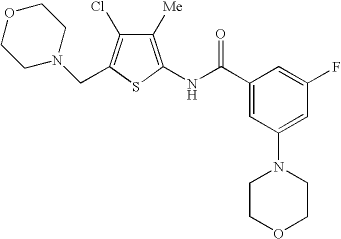

To a solution of the product of Example 1F, N-[4-Chloro-3-methyl-5-(morpholine-4-carbothioyl)-thiophen-2-yl]-3-fluoro-5-morpholin-4-yl-benzamide, (50 mg, 0.11 mmol) in dry THF (4 ml) was added methyl iodide (176 mg, 1.24 mmol). The reaction mixture was stirred at room temperature overnight and evaporated to dryness under reduced pressure. The resulting dark orange crystalline residue was re-dissolved in dry methanol (3 ml) and treated with sodium borohydride (5 mg, 0.13 mmol) and stirred at room temperature for 3 hours. The reaction mixture was diluted with 1N sodium hydroxide (8 ml) and extracted with dichloromethane. The organics were combined, washed with brine solution, dried (MgSO₄) and evaporated to dryness under reduced pressure. Purification by preparative HPLC gave the title compound as an off-white solid (28 mg, 61%); LC MS M+H 454

### Example 2

### Preparation of 1-[5-tert-butyl-2(4-fluoro-phenyl)-2H-pyrazol-3-yl]-3-(4-chloro-3-methyl-5-morpholin-4-ylmethyl-thiophen-2-yl)urea

### 2A. Preparation of (3-chloro-4-methyl-thiophen-2-yl)-morpholin-4-yl-methanone

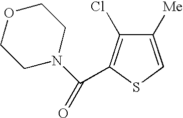

To a solution of 3-chloro-4-methyl-thiophen-2-carboxylic acid (20 g, 11.3 mmol) in dichloromethane (450 ml) was added EDAC (25.6 g, 13 mmol), HOBt (20 g, 13 mmol) followed by morpholine (10 ml, 12 mmol). The reaction mixture was stirred at room temperature overnight and then diluted with dichloromethane (500 ml). The diluted reaction mixture washed with 5% citric acid solution (300 ml) and brine (300 ml), dried (MgSO₄), filtered and the solvent was removed under reduced pressure to afford the title compound as a crude product (˜23 g) which was used immediately in the next step without purification). LC MS M+H 246

### 2B. Preparation of (5-amino-3-chloro-4-methyl-thiophen-2-yl)-morpholin-4-yl-methanone

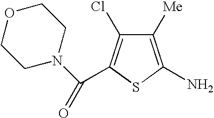

To a solution of (3-chloro-4-methyl-thiophen-2-yl)-morpholin-4-yl-methanone (Example 2B) (9.4 g, 38 mmol) in acetonitrile (600 ml) was added nitronium tetrafluoroborate (80 mmol) at 0° C. The reaction mixture was allowed to warm to room temperature over 18 hours, then diluted with water (700 ml) and extracted with dichloromethane (900 ml). The organic solution washed with saturated sodium bicarbonate solution (500 ml) and brine (500 ml), dried (MgSO₄), filtered and the solvent removed under reduced pressure to afford the crude product (11 g) as an orange oil, which was used immediately in the next step without purification.

To a solution of the crude product (11 g, 3.7 mmol) in dioxane (250 ml) and water (50 ml) was added iron powder (19 g) followed by iron sulphate heptahydrate (23 g). The reaction mixture was then heated to reflux for 4 hours before cooling to room temperature. The solvent was then removed under reduced pressure and the residue was subjected to purification by flash column chromatography on silica gel, eluting with ethyl acetate/petroleum ether mixtures to afford the title compound as a brown oil (7.7 g); LC MS M+H 261. This was used immediately in the urea formation reaction of Example 2D.

### 2C. Preparation of 5-tert-butyl-2-(4-fluoro-phenyl)-2H-pyrazol-3-ylamine 5

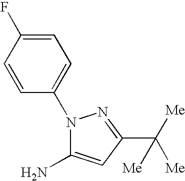

The title compound is commercially available from Butt Park of Bath, UK or can be prepared according to the following method.

To a stirred solution of 4-fluorophenylhydrazine hydrochloride (30 g, 111.5 mmol) in EtOH (800 ml) was pivolylacetonitrile (1 equiv.) and the reaction mixture was heated at reflux for 10 hours. After cooling to room temperature, the solvent was removed under reduced pressure and the residue was subjected to purification by trituration with diethyl ether/ethyl acetate mixtures to afford the title compound as a pale brown solid (27.6 g). LC MS M+H 234

### 2D. Preparation of 1-[5-tert-butyl-2-(4-fluoro-phenyl)-2H-pyrazol-3-yl]-3-[4-chloro-3-methyl-5-(morpholine-4-carbonyl)-thiophen-2-yl]urea

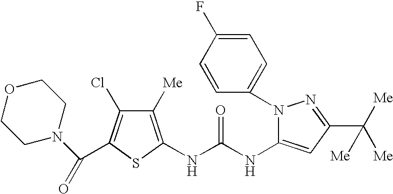

To a stirred solution of (5-amino-3-chloro-4-methyl-thiophen-2-yl)-morpholin-4-yl-methanone (Example 2B) (8 g) in dry dichloromethane (350 ml) was added 20% phosgene in toluene (65 ml) at room temperature and the reaction mixture was stirred for 18 hrs to allow formation of the isocyanate to go to completion. The solvent was removed under reduced pressure and the residue was re-dissolved in dry dichloromethane (300 ml) and treated with 5-tert-butyl-2-(4-fluoro-phenyl)-2H-pyrazol-3-yl-amine (Example 2C) dropwise in dry dichloromethane (80 ml) and the reaction mixture was then stirred at room temperature for 24 hours. The reaction mixture was quenched with saturated sodium hydrogen carbonate and extracted with dichloromethane (x3). The organics were combined, washed with 2N HCl, saturated brine solution, dried (MgSO₄), filtered and the solvent removed under reduced pressure. The residue was subjected to purification by flash column chromatography on silica gel, eluting with 5% methanol/dichloromethane to afford the title compound as a solid (10.3 g); LC MS M+H 520.

### 2E. Preparation of 1-[5-tert-butyl-2-(4-fluoro-phenyl)-2H-pyrazol-3-yl]-3-[4-chloro-3-methyl-5-(morpholine-4-carbothioyl)-thiophen-2-yl]urea

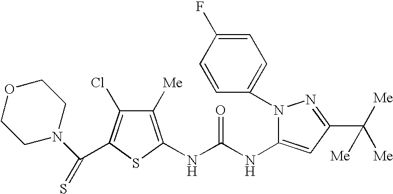

To a stirred solution of 1-[5-tert-butyl-2-(4-fluoro-phenyl)-2H-pyrazol-3-yl]-3-[4-chloro-3-methyl-5-(morpholine-4-carbonyl)-thiophen-2-yl]urea (Example 2D) (2 g, 3.85 mmol) in dry THF was added Lawesson's reagent (1.87 g, 4.6 mmol) and the reaction mixture was stirred at room temperature overnight. The solvent was removed under reduced pressure and the residue was subjected to purification by flash column chromatography on silica gel, eluting with 1:1 ethyl acetate/hexanes to afford the title compound as a solid (1.58 g); LC MS M+H 536.

### 2F. Preparation of 1-[5-tert-butyl-2(4-fluoro-phenyl)-2H-pyrazol-3-yl]-3-(4-chloro-3-methyl-5-morpholin-4-ylmethyl-thiophen-2-yl)urea

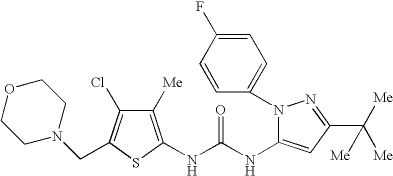

To a stirred solution of (1.58 g, 2.95 mmol) in dry THF (150 ml) was added methyl iodide (1.9 ml, 15 equiv.) and the reaction mixture was stirred at 50° C. overnight. The solvent was removed under reduced pressure and the residue was re-dissolved in dry methanol (100 ml) and treated with sodium borohydride (140 mg, 1.05 equiv.). The reaction mixture was then stirred at room temperature for 3 hours before diluting with 1N NaOH (100 ml) and extracting with ethyl acetate (x3). The organic solutions were combined and then washed with brine, dried (MgSO₄), filtered and the solvent was removed under reduced pressure to afford the crude product as a dark orange solid. Purification by flash column chromatography on silica gel, eluting with 3:1 ethyl acetate/hexanes, gave the title compound as an off-white solid (0.76 g); LC MS M+H 506.

### Example 3

### 1-[5-tert-Butyl-2-(2,4-difluoro-phenyl)-2H-pyrazol-3-yl]-3-(4-chloro-3-methyl-5-morpholin-4-ylmethyl-thiophen-2-yl)-urea

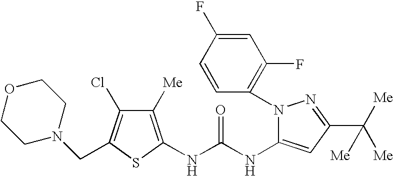

The title compound was prepared from (5-amino-3-chloro-4-methyl-thiophen-2-yl)-morpholin-4-yl-methanone (Example 2B) and 5-tert-butyl-2-(2,4-difluorophenyl)-2H-pyrazol-3-ylamine following the procedures described in Example 2. LC MS M+H 524

### Example 4

### 1-(4-Chloro-3-methyl-5-morpholin-4-ylmethyl-thiophen-2-yl)-3-[5-(tetrahydro-furan-2-yl)-[13,4]thiadiazol-2-yl]-urea

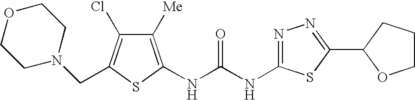

The title compound was prepared from (5-amino-3-chloro-4-methyl-thiophen-2-yl)-morpholin-4-yl-methanone (Example 2B) and 5-(tetrahydro-furan-2-yl)-[1,3,4]thiadiazole-2-ylamine (commercially available) following the procedures described in Example 2. LC MS M+H 444

Biological Activity

### Example 5

p38 MAP Kinase Inhibitory Activity

Measurement of p38 MAP Kinase Inhibitory Activity (IC₅₀)

Compounds of the invention were tested for p38 MAP kinase inhibitory activity using the protocol set out below.

In the assay, an inactive a isoform of p38 mitogen-activated protein kinase was used. The structure of this kinase at 2,1-A resolution is described in the article by Wang Z, Harkins P C, Ulevitch R J, Han J, Cobb M H and Goldsmith E J. in Proc. Natl. Acad. Sci. USA 1997 Mar. 18; 94(6):2327. The α isoform of p38 MAP kinase was activated using the MKK6 kinase obtained from Upstate Biotechnology. The selective activation of p38 mitogen-activated protein (MAP) kinase isoforms by the MAP kinase kinase MKK6 is described in the article by Enslen, H; Raingeaud, J and Davis, R J in The Journal of Biological Chemistry, Volume 273, Issue 3, Jan. 16, 1998, Pages 1741-1748.

The protocol was as follows:

1 ml of fresh assay buffer (25 mM HEPES pH 7.4, 25 mM β-glycerophosphate, 5 mM EDTA, 15 mM MgCl₂, 100 μM ATP, 1 mM sodium orthovanadate, 1 mM DTT), 35 μg of inactive purified α p38 and 0.12 μg of active MKK6 (1688 U/mg—Upstate Biotechnology) are mixed and incubated at room temperature overnight to activate the p38. The activated p38 is then diluted sixfold with assay buffer without ATP, and 10 μl mixed with 5 μl of various dilutions of the test compound in DMSO (up to 1.7%) in a 96 well plate and incubated at room temperature for 1.5 hours. Next, 10 μl of MBP mix (150 μl 10× strength assay buffer (250 mM HEPES pH 7.4, 250 mM β-glycerophosphate, 50 mM EDTA, 150 mM MgCl₂), 1.5 μl of 10 mM DTT & 10 mM sodium orthovanadate, 17.5 μl of 10 mM ATP, 713 μl H₂O, 35 μCi γ³³P-ATP, 100 μl of myelin basic protein (MBP) (5 mg/ml)) is added to each well. MBP is a protein of bovine origin having a molecular weight of 18.4 kDa and is obtained from Upstate Biotechnology. The reaction is allowed to proceed for 50 minutes before being stopped with an excess of ortho-phosphoric acid (5 μl at 12.5%).

γ³³P-ATP which remains unincorporated into the myelin basic protein is separated from phosphorylated MBP on a Millipore MAPH filter plate. The wells of the MAPH plate are wetted with 0.5% orthophosphoric acid, and then the results of the reaction are filtered with a Millipore vacuum filtration unit through the wells. Following filtration, the residue is washed twice with 200 μl of 0.5% orthophosphoric acid. Once the filters have dried, 25 μl of Microscint 20™ scintillant is added, and then counted on a Packard Topcount for 30 seconds. The % inhibition of the p38 activity is calculated and plotted in order to determine the concentration of test compound required to inhibit 50% of the p38 activity (IC₅₀).

The compounds of Examples 1 to 4 were tested using the assay and all were found to inhibit p38 activity. All of the compounds had IC₅₀ values of less than 5 μM.

### Example 6

Inhibition of LPS-Induced TNF-α Production in THP-1 Cells. In Vitro Assay

The ability of the compounds of this invention to inhibit the TNF-α release may be determined using a minor modification of the methods described in Rawlins P., et al., “Inhibition of endotoxin-induced TNF-α production in macrophages by 5Z-7-oxo-zeaenol and other fungal resorcyclic acid lactones,” International J. of Immunopharmacology, 21, 799, (1999).

THP-1 cells, human monocytic leukaemic cell line, ECACC) are maintained in culture medium [RPMI 1640 (Invitrogen) and 2 mM L-Glutamine supplemented with 10% foetal bovine serum (Invitrogen)] at approximately 37° C. in humidified 5% CO₂ in stationary culture.

THP-1 cells are suspended in culture medium containing 50 ng/ml PMA (SIGMA), seeded into a 96-well tissue culture plate (IWAKI) at 1×10⁵ cells/well (100 μl/well) and incubated as described above for approximately 48 h. The medium is then aspirated, the wells washed twice in Phosphate Buffered Saline and 1 μg/ml LPS (SIGMA) in culture medium is added (200%/well).

Test compounds are reconstituted in DMSO (SIGMA) and then diluted with the culture medium such that the final DMSO concentration is 0.1%. Twenty microlitre aliquots of test solution or medium only with DMSO (solvent control) are added to triplicate wells immediately following LPS addition, and incubated for 6 h as described above. Culture supernatants are collected and the amount of human TNF-α present is determined by ELISA (R&D Systems) performed according to the manufacturer's instructions.

The IC₅₀ is defined as the concentration of the test compound corresponding to half maximal inhibition of the control activity by non-linear regression analysis of their inhibition curves.

### Example 7

Measurement of C-raf kinase Inhibitory Activity (IC₅₀)

Human c-raf (Upstate) is diluted to a 10x working stock in 50 mM Tris pH 7.5, 0.1 mM EGTA, 0.1 mM sodium vanadate, 0.1% P-mercaptoethanol, 1 mg/ml BSA. One unit equals the incorporation of 1 nmol of phosphate per minute into myelin basic protein per minute.

In a final reaction volume of 25 μl, c-raf (5-10 mU) is incubated with 25 mM Tris pH 7.5, 0.02 mM EGTA, 0.66 mg/ml myelin basic protein, 10 mM MgAcetate, [γ-³³P-ATP] (specific activity approx 500 cpm/μmol, concentration as required) and appropriate concentrations of inhibitor or diluent as control. The reaction is initiated by the addition of Mg²+[γ-³³P-ATP]. After incubation for 40 minutes at room temperature the reaction is stopped by the addition of 5 μl of a 3% phosphoric acid solution. 10 μl of the reaction mixture is spotted onto a P30 filtermat and washed 3 times for 5 minutes in 75 mM phosphoric acid and once in methanol prior to drying and counting to determine the C-raf activity.

The % inhibition of the C-raf kinase activity is calculated and plotted in order to determine the concentration of test compound required to inhibit 50% of the C-raf kinase activity (IC₅₀).

The compounds of Examples 1 and 4 have been found to have IC₅₀ values of less than 25 μM, and the compounds of Examples 2 and 3 have been found to have IC₅₀ values of less than 1 μM.

### Example 8

Xenograft Studies

The anti-cancer properties of the compounds of the invention can be determined using Xenograph studies. The studies can be used to determine the effects of test compound on the rate of body weight loss induced by C26 tumours in normal mice, the anti-tumour effect and generate tissues for evaluation of biomarkers.

Protocol

Male Balb c mice (4-5 weeks of age) are implanted with mouse C26 tumour fragments subcutaneously in the region of the right axilla (day 0). Treatment begins when tumours reach 150 mg and the animals have been grouped such that tumour weight and body weight has a mean intergroup variation of <10%. Animals are then dosed twice daily at 8 and 16 hr intervals by the intravenous route with either test compound in vehicle, or with vehicle alone. The vehicle is 10% DMS0: 20% PEG200: 70% hydroxypropyl beta-cyclodextrin (25% w/v in water) adjusted to between pH4-8 as necessary with NaOH. The dose volume is 10 ml/kg. The study is conducted in 2 parts. Firstly, the maximum tolerated dose (MTD) is determined in groups of 3 mice (tumour starting volumes 100 mg). Secondly, the effects of test substance on body weight loss and tumour burden is determined in groups of 12 mice at doses which are fractions of the MTD (likely to be in the range 1-100 mg/kg). The dosing period may be extended as required to allow appropriate levels of statistical significance to develop in measurements between groups of control and test animals. Measurements taken throughout the study include tumour burden and body weight (measured three times per week). Food consumption may also be measured. Change in body weight and tumour volume over time, are used to monitor progress of the study, and form the clinical endpoints. Tumour and serum samples may be further investigated for biomarker profiles, e.g. cytokines, and/or determination of compound concentration. The study protocol may be based on methodology described variously by Strassmann et al. (Strassmann et al., 1992, J Clin Invest, 89, 1681-1684; Strassmann et al., 1993, J Clin Invest, 92, 2152-2159; Strassmann et al. 1993, Cytokine, 5(5), 463-468).

### Example 9

Kinase assay of Tie2, VEGFR2, EPHB2, FGFR-3 Assays for activity against the above kinases can be carried out using out using the proprietary 33 PanQinase® Activity Assay provided by Proqinase GmbH, of Freiburg, Germany. The assay is performed in 96 well FlashPlates™ (PerkinElmer). The reaction cocktail (50 μl final volume) is composed of; 20 μl assay buffer (final composition 60 mM HEPES-NaOH, pH 7.5, 3 mM MgCl₂, 3 μM Na-orthovanadate, 1.2 mM DTT, 50 μg/ml PEG₂₀₀₀, 5 μl ATP solution (final concentration 1 μM [□-³³P]-ATP (approx 5×10⁵ cpm per well)), 5 μl test compound (in 10% DMSO), 10 μl substrate/10 μl enzyme solution (premixed). The final amounts of enzyme and substrate used are as set out below.

| | Kinase | | Substrate |
|---|---|---|---|
| Kinase | ng/50 μl | Substrate | ng/50 μl |
| Tie2 | 200 | Poly (Glu, Tyr) 4:1 | 125 |
| VEGF-R2 | 25 | Poly (Glu, Tyr) 4:1 | 125 |
| EPHB2 | 200 | Poly (Ala, Glu, Lys, Tyr) 6:2:5:1 | 125 |
| FGFR-3 | 100 | Poly(Glu:Tyr) 4:1 | 125 |

The reaction cocktails are incubated at 30° C. for 80 minutes. The reaction is tehn stopped with 50 μl of 2% H₃PO₄, plates are aspirated and washed twice with 200 μl 0.9% NaCl. Incorporation of ³³Pi is determined with a microplate scintillation counter. Background values are subtracted from the data before calculating the residual activities for each well. IC₅₀ values are calculated using Prism 3.03.

Pharmaceutical Formulations

### Example 10

(i) Tablet Formulation

A tablet composition containing a compound of the formula (I) is prepared by mixing 50 mg of the compound with 197 mg of lactose (BP) as diluent, and 3 mg magnesium stearate as a lubricant and compressing to form a tablet in known manner.

(ii) Capsule Formulation

A capsule formulation is prepared by mixing 100 mg of a compound of the formula (I) with 100 mg lactose and filling the resulting mixture into standard opaque hard gelatin capsules.

(iii) Injectable Formulation I

A parenteral composition for administration by injection can be prepared by dissolving a compound of the formula (I) (e.g. in a salt form) in water containing 10% propylene glycol to give a concentration of active compound of 1.5% by weight. The solution is then sterilised by filtration, filled into an ampoule and sealed.

(iv) Injectable Formulation II

A parenteral composition for injection is prepared by dissolving in water a compound of the formula (I) (e.g. in salt form) (2 mg/ml) and mannitol (50 mg/ml), sterile filtering the solution and filling into sealable 1 ml vials or ampoules.

(v) Subcutaneous Injection Formulation

A composition for sub-cutaneous administration is prepared by mixing a compound of the formula (I) with pharmaceutical grade corn oil to give a concentration of 5 mg/ml. The composition is sterilised and filled into a suitable container.

(vi) Aerosol Formulation

An aerosol formulation for administration by inhalation is prepared by weighing micronised compound of the formula (I) (60 mg) directly into an aluminium can and then adding 1,1,1,2-tetrafluorethane (to 13.2 g) from a vacuum flask. A metering valve is crimped into place and the sealed can is sonicated for five minutes. The resulting formulation delivers the compound of formula (I) as an aerosol in an amount of 250 mg of per actuation.

### Equivalents

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1. A compound of the formula (I): 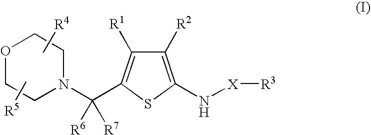
or a salt, solvate or N-oxide thereof, wherein:
R¹ and R² are the same or different and each is selected from hydrogen, saturated C₁₋₃ hydrocarbyl, halogen and cyano;
X is selected from C═O, C═S, C(═O)NH, C(═S)NH, C(═O)O, C(═O)S, C(═S)O and C(═S)S;
R³ is selected from aryl and heteroaryl groups each having from 5 to 12 ring members and being unsubstituted or substituted by one or more substituent groups R¹⁰;
R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}—R^{e} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
or two adjacent groups R¹⁰, together with the carbon atoms or heteroatoms to which they are attached may form a 5-membered heteroaryl ring or a 5- or 6-membered non-aromatic heterocyclic ring, wherein the said heteroaryl and heterocyclic groups contain up to 3 heteroatom ring members selected from N, O and S;
R^{c} is selected from hydrogen and C₁₋₄ hydrocarbyl; and
X¹ is O, S or NR^{c} and X² is ═O, ═S or ═NR^{c};
R⁴ and R⁵ are the same or different and are selected from hydrogen and methyl;
or one of R⁴ and R⁵ is selected from hydroxymethyl and ethyl and the other is hydrogen; and
R⁶ and R⁷ are the same or different and are selected from hydrogen and methyl.

2. A compound according to claim 1 wherein R³ is a monocyclic aryl or heteroaryl group.

3-82. (canceled)

83. A compound according to claim 2 wherein the aryl group or heteroaryl group R³ contains one or more substituent groups R¹⁰ selected from halogen, carbocyclic and heterocyclic groups having from 4 to 7 ring members and optionally substituted C₁₋₈ hydrocarbyl groups.

84. A compound according to claim 83 wherein the group R³ contains a substituent R¹⁰ which is a carbocyclic or heterocyclic group having from 4 to 7 ring members and said carbocyclic or heterocyclic group is linked to the aryl or heteroaryl ring via a carbon nitrogen bond.

85. A compound according to claim 84 wherein the carbocyclic or heterocyclic group R¹⁰ is a 4 to 7 membered heterocyclic group R⁸ selected from morpholine, piperidino, piperazino, N-methyl piperazino, tetrahydrofuranyl and pyrrolidino.

86. A compound according to claim 1 wherein X is C═O or C(═O)NH.

87. A compound according to claim 1 wherein R¹ is selected from hydrogen, saturated C₁₋₃ hydrocarbyl and halogen.

88. A compound according to claim 1 wherein R² is selected from hydrogen, saturated C₁₋₃ hydrocarbyl and halogen.

89. A compound according to claim 87 wherein R¹ is chlorine.

90. A compound according to claim 88 wherein R² is methyl.

91. A compound according to claim 1 wherein R⁴ and R⁵ are both hydrogen.

92. A compound according to claim 1 wherein R⁶ and R⁷ are both hydrogen.

93. A compound according to claim 1 which is selected from:
N-(4-chloro-3-methyl-5-(morpholin-ylmethyl-thiophen-2-yl)-3-fluoro-morpholin-4-yl-benzamide;
1-[5-tert-butyl-2(4-fluoro-phenyl)-2H-pyrazol-3-yl]-3-(4-chloro-3-methyl-5-morpholin-4-ylmethyl-thiophen-2-yl)urea;
1-[5-tert-butyl-2-(2,4-difluoro-phenyl)-2H-pyrazol-3-yl]-3-(4-chloro-3-methyl-5-morpholin-4-ylmethyl-thiophen-2-yl)-urea; and
1(4-chloro-3-methyl-5-morpholin-4-ylethyl-thiophen-2-yl)-3-[5-(tetrahydro-furan-2-yl)-[1,3,4]thiadiazol-2-yl]-urea.

94. A pharmaceutical composition comprising a compound of the formula (I): 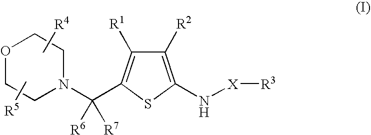
or a salt, solvate or N-oxide thereof, wherein:
R¹ and R² are the same or different and each is selected from hydrogen, saturated C₁₋₃ hydrocarbyl, halogen and cyano;
X is selected from C═O, C═S, C(═O)NH, C(═S)NH, C(═O)O, C(═O)S, C(═S)O and C(═S)S;
R³ is selected from aryl and heteroaryl groups each having from 5 to 12 ring members and being unsubstituted or substituted by one or more substituent groups R¹⁰;
R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}—R^{e} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; or two adjacent groups R¹⁰, together with the carbon atoms or heteroatoms to which they are attached may form a 5-membered heteroaryl ring or a 5- or 6-membered non-aromatic heterocyclic ring, wherein the said heteroaryl and heterocyclic groups contain up to 3 heteroatom ring members selected from N, O and S;
R^{c} is selected from hydrogen and C₁₋₄ hydrocarbyl; and
X¹ is O, S or NR^{c} and X² is ═O, ═S or ═NR^{c};
R⁴ and R⁵ are the same or different and are selected from hydrogen and methyl;
or one of R⁴ and R⁵ is selected from hydroxymethyl and ethyl and the other is hydrogen; and
R⁶ and R⁷ are the same or different and are selected from hydrogen and methyl;
together with a pharmaceutically acceptable carrier.

95. A method for the prophylaxis or treatment of a disease state or condition mediated by a p38 MAP kinase, wherein the disease state or condition mediated by a p38 MAP kinase is selected from:
(i) inflammatory and arthritic diseases and conditions, Reiter's syndrome, acute synovitis, rheumatoid arthritis, osteoarthritis, rheumatoid spondylitis, gouty arthritis, traumatic arthritis, rubella arthritis, psoriatic arthritis, graft vs. host reaction and allograft rejections;
(ii) chronic inflammatory lung diseases, emphysema, chronic pulmonary inflammatory disease, chronic obstructive pulmonary disease (COPD), adult respiratory distress syndrome and acute respiratory distress syndrome (ARDS);
(iii) lung diseases and conditions, tuberculosis, silicosis, pulmonary sarcoidosis, pulmonary fibrosis and bacterial pneumonia;
(iv) inflammatory diseases and conditions of the enteric tract, inflammatory bowel disease, Crohn's disease and ulcerative colitis;
(v) toxic shock syndrome and related diseases and conditions, sepsis, septic shock, endotoxic shock, gram negative sepsis and the inflammatory reaction induced by endotoxin;
(vi) Alzheimer's disease;
(vii) reperfusion injury;
(vii) diseases and conditions selected from atherosclerosis; muscle degeneration; gout; cerebral malaria; bone resorption diseases; fever and myalgias due to infection, influenza; cachexia, cachexia secondary to infection or malignancy, cachexia secondary to acquired immune deficiency syndrome (AIDS); AIDS; ARC (AIDS related complex); keloid formation; scar tissue formation; pyresis and asthma; which method comprises administering to a subject in need thereof a compound of the formula (I): 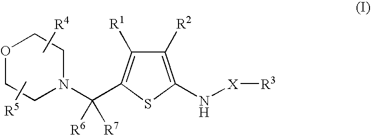
or a salt, solvate or N-oxide thereof, wherein:
R¹ and R² are the same or different and each is selected from hydrogen, saturated C₁₋₃ hydrocarbyl, halogen and cyano;
X is selected from C═O, C═S, C(═O)NH, C(═S)NH, C(═O)O, C(═O)S, C(═S)O and C(═S)S;
R³ is selected from aryl and heteroaryl groups each having from 5 to 12 ring members and being unsubstituted or substituted by one or more substituent groups R¹⁰;
R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; or two adjacent groups R¹⁰, together with the carbon atoms or heteroatoms to which they are attached may form a 5-membered heteroaryl ring or a 5- or 6-membered non-aromatic heterocyclic ring, wherein the said heteroaryl and heterocyclic groups contain up to 3 heteroatom ring members selected from N, O and S;
R^{c} is selected from hydrogen and C₁₋₄ hydrocarbyl; and
X¹ is O, S or NR^{c} and X² is ═O, ═S or ═NR^{c};
R⁴ and R⁵ are the same or different and are selected from hydrogen and methyl;
or one of R⁴ and R⁵ is selected from hydroxymethyl and ethyl and the other is hydrogen; and
R⁶ and R⁷ are the same or different and are selected from hydrogen and methyl.

96. A method according to claim 95 wherein the disease state or condition is selected from inflammatory diseases and conditions, rheumatoid arthritis and osteoarthritis.

97. A method according to claim 95 wherein the disease state or condition is chronic obstructive pulmonary disease (COPD).

98. A method for treating a disease or condition comprising or arising from abnormal cell growth in a mammal, the method comprising administering to the mammal a therapeutically effective amount of a compound of the formula (I): 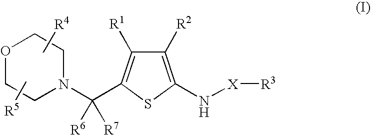
or a salt, solvate or N-oxide thereof, wherein:
R¹ and R² are the same or different and each is selected from hydrogen, saturated C₁₋₃ hydrocarbyl, halogen and cyano;
X is selected from C═O, C═S, C(═O)NH, C(═S)NH, C(═O)O, C(═O)S, C(═S)O and C(═S)S;
R³ is selected from aryl and heteroaryl groups each having from 5 to 12 ring members and being unsubstituted or substituted by one or more substituent groups R¹⁰;
R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; or two adjacent groups R¹⁰, together with the carbon atoms or heteroatoms to which they are attached may form a 5-membered heteroaryl ring or a 5- or 6-membered non-aromatic heterocyclic ring, wherein the said heteroaryl and heterocyclic groups contain up to 3 heteroatom ring members selected from N, O and S;
R^{c} is selected from hydrogen and C₁₋₄ hydrocarbyl; and
X¹ is O, S or NR^{c} and X² is ═O, ═S or ═NR^{c};
R⁴ and R⁵ are the same or different and are selected from hydrogen and methyl; or one of R⁴ and R⁵ is selected from hydroxymethyl and ethyl and the other is hydrogen; and
R⁶ and R⁷ are the same or different and are selected from hydrogen and methyl.

99. A method for the prophylaxis or treatment of a disease state or condition mediated by a raf kinase, which method comprises administering to a subject in need thereof a compound of the formula (I): 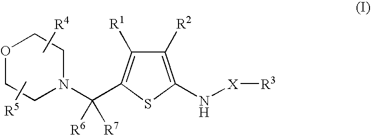
or a salt, solvate or N-oxide thereof, wherein:
R¹ and R² are the same or different and each is selected from hydrogen, saturated C₁₋₃ hydrocarbyl, halogen and cyano;
X is selected from C═O, C═S, C(═O)NH, C(═S)NH, C(═O)O, C(═O)S, C(═S)O and C(═S)S;
R³ is selected from aryl and heteroaryl groups each having from 5 to 12 ring members and being unsubstituted or substituted by one or more substituent groups R¹⁰;
R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; or two adjacent groups R¹⁰, together with the carbon atoms or heteroatoms to which they are attached may form a 5-membered heteroaryl ring or a 5- or 6-membered non-aromatic heterocyclic ring, wherein the said heteroaryl and heterocyclic groups contain up to 3 heteroatom ring members selected from N, O and S;
R^{c} is selected from hydrogen and C₁₋₄ hydrocarbyl; and
X¹ is O, S or NR^{c} and X² is ═O, ═S or ═NR^{c};
R⁴ and R⁵ are the same or different and are selected from hydrogen and methyl; or one of R⁴ and R⁵ is selected from hydroxymethyl and ethyl and the other is hydrogen; and
R⁶ and R⁷ are the same or different and are selected from hydrogen and methyl.

100. A process for the preparation of a compound of the formula (I): 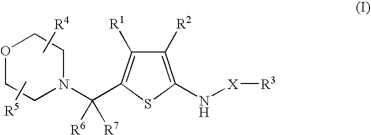
or a salt, solvate or N-oxide thereof, wherein:
R¹ and R² are the same or different and each is selected from hydrogen, saturated C₁₋₃ hydrocarbyl, halogen and cyano;
X is selected from C═O, C═S, C(═O)NH, C(═S)NH, C(═O)O, C(═O)S, C(═S)O and C(═S)S;
R³ is selected from aryl and heteroaryl groups each having from 5 to 12 ring members and being unsubstituted or substituted by one or more substituent groups R¹⁰;
R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; or two adjacent groups R¹⁰, together with the carbon atoms or heteroatoms to which they are attached may form a 5-membered heteroaryl ring or a 5- or 6-membered non-aromatic heterocyclic ring, wherein the said heteroaryl and heterocyclic groups contain up to 3 heteroatom ring members selected from N, O and S;
R^{c} is selected from hydrogen and C₁₋₄ hydrocarbyl; and
X¹ is O, S or NR^{c} and X² is ═O, ═S or ═NR^{c};
R⁴ and R⁵ are the same or different and are selected from hydrogen and methyl; or one of R⁴ and R⁵ is selected from hydroxymethyl and ethyl and the other is hydrogen; and
R⁶ and R⁷ are the same or different and are selected from hydrogen and methyl;
which process comprises the S-alkylation of a compound of the formula (X): 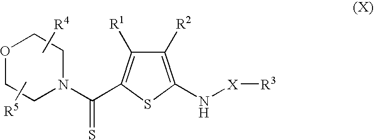
using an alkylating agent to give a thioimidate intermediate followed by:
(i) reduction of the thioimidate intermediate to give a compound of formula (I) in which R⁶ and R⁷ are hydrogen by means of a reducing agent; or
(ii) treating the thioimidate intermediate with methyl lithium or a methyl Grignard reagent, followed by a reducing agent to give a compound of the formula (I) in which one of R⁶ and R⁷ is methyl; or
(iii) treating the thioimidate intermediate with more than one equivalent of methyl lithium or a methyl Grignard reagent to give a compound of the formula (I) in which both R⁶ and R⁷ are methyl.
